(19) United States
(12) Patent Application Publication
(10) Pub. No.: US 2008/0070311 A1
(43) Pub. Date: Mar. 20, 2008

(54) Microfluidic flow cytometer and applications of same

(75) Inventor: Dongqing Li, Antioch, TN (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Vanderbilt University, Nashville, TN (US)

(21) Appl. No.: 11/523,796

(22) Filed: Sep. 19, 2006

(51) Int. Cl. G01N 33/48; G01N 27/00

(52) U.S. Cl. 436/63; 204/601

(57) ABSTRACT

A flow cytometer. In one embodiment the flow cytometer includes a microchannel structure adapted for transporting a fluid medium containing one or more types of particles; means for generating electrokinetically microfluidic flows to transport the fluid medium in the microchannel structure so as to differentiate the one or more types of particles of the fluid medium therein; and an optical detection system coupled with the microchannel structure for detecting the differentiated one or more types of particles of the fluid medium.

### CROSS-REFERENCE TO RELATED PATENT APPLICATION

This application is related to a co-pending U.S. patent application entitled “DC-Dielectrophoresis Microfluidic Apparatus and Applications of Same,” by Dongqing Li with Attorney Docket No. 14506-55547, filed Sep. 19, 2006, which has the same assignee as the present application and has been concurrently filed herewith. The applicant of that application is also applicant of this application. The disclosure of the above-identified co-pending application is incorporated in its entirety herein by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [25] represents the 25th reference cited in the reference list, namely, Ye C, Xuan X, Li D. Eccentric electrophoretic motion of a sphere in circular cylindrical microchannels. Microfluidics and Nanofluidics. 2005; 1:234-41.

### FIELD OF THE INVENTION

The present invention relates generally to a flow cytometer, and more particularly to a microfluidic flow cytometer and applications of same.

### BACKGROUND OF THE INVENTION

Flow cytometry provides a method of detecting and analyzing particles contained in a sample, for example, blood cells in blood such as red blood cells (erythrocytes), white blood cells (leukocytes) and blood platelets (thrombocytes), or material components in urine such as bacteria, blood cells, white blood cells, epithelial cells or casts. These cells or material components may increase or decrease in number in accordance with a disease. Accordingly, a disease can be diagnosed by detecting the status of each cell or material component on the basis of information about granules or particles in the sample.

For example, in the field of HIV treatments, a single most important parameter for disease staging is the number of the CD4+ T cell (in the unit of cells/mm³) in peripheral blood. However, the laboratory evaluation of CD4+ T cell numbers can be cumbersome and expensive. Typically, the total lymphocyte count is determined by a routine CBC (complete blood count) assay, the percentage of CD4+ T lymphocytes as a function of total lymphocytes is determined by a flow cytometry, and these values are multiplied to determine an absolute CD4+ T cell number.

Usually, a benchtop flow cytometry is very expensive and needs to be careful maintained. The operation of such a benchtop flow cytometer requires specially trained personnel. Additionally, the sample volumes consumed by the benchtop flow cytometry are very large, typically in a range of hundred microliters to several hundred microliters.

Recently, efforts have been made to apply microfluidic technologies to flow cytometric analysis of cells, which may lead to develop cost effective, small sized and portable flow cytometers. For example, Tung et al. [1] presented a flow cytometer chip using polydimethylsiloxane (PDMS) for fluorescence-labeled particle detection using a two-color, multi-angle detection system via embedded fibers. The size of the flow cytometer is significantly reduced. However, the flow cytometer chip lacks portability as it requires a manually operated external liquid handling system, e.g., two syringe pumps and tubing, to focus a cell-carrying stream in a detection channel. Fu et al. [2] reported a flow cytometer chip using electrokinetically microfluidic flow focusing mechanism. The flow cytometer chip has a glass plate with a pair of embedded optical fibers for counting particles moving through a microchannel. All these flow cytometers have only one function, i.e. counting the number of single-sized particles. However, a practical flow cytometer must be able to handle mixtures of diverse cells that must be differentiated and counted by size and by their fluorescent dye tags. Additionally, using embedded waveguides or optical fibers on each side of the detection channel of a flow cytometer requires large lateral space and thus prevents such a chip from having multiple parallel channels, which increases the throughput of the device. Furthermore, using the embedded waveguides or optical fibers significantly also increases the cost of the flow cytometers. A microchip based CD4 counting system was also reported in [3]. However, it requires a large external liquid delivery system including a pump, tubing and valves, an external membrane filter for cell separation, and a conventional optical detection system. The system is manually operated with complicated procedures, and is not portable.

Therefore, a heretofore unaddressed need exists in the art to address the aforementioned deficiencies and inadequacies.

### SUMMARY OF THE INVENTION

The present invention, in one aspect, relates to a flow cytometer that can be used for counting and differentiating particles in a liquid medium of interest. The liquid medium of interest may comprise a biological fluid of a living subject. The biological fluid includes blood or urine. The blood or urine comprises one or more types of particles or cells. The one or more types of cells are differentiatable by their sizes, functions or a combination of them. The one or more types of cells may comprise CD4+ cells, and/or CD3+ cells. In one embodiment, the CD4+ cells and CD3+ cells are labeled with a first and second antibodies, respectively, where the first and second antibodies are excited with light of different wavelengths. The one or more types of cells are associated with a disease. By detecting and differentiating the one or more types of cells that are associated with a disease, the disease may be detected or identified, and/or treated.

In one embodiment, the flow cytometer comprises a first substrate having a first surface and an opposite, second surface defining a body portion therebetween. The flow cytometer further comprises a microchannel structure formed in the body portion of the first substrate for differentiating particles in a liquid medium of interest. The microchannel structure includes a first particle separation unit, a second particle separation unit, and a flow focusing unit.

In one embodiment, each of the first and second particle separation units has a first and second inlet ports, a first and second outlet ports, and a first, second and third microchannels. Each of the first, second and third microchannels is formed with a first open end, an opposite, second open end, and a first side wall and an opposite, second side wall defining a corresponding channel width, w1, w2, w3, therebetween, respectively. Each channel width is in a range of about 0.1-1,000 μm, preferable in a range of about 1-500 μm. The first microchannel is in fluid communication with the first inlet port and the second microchannel through the first and second open ends, respectively, thereby forming a first junction of the first and second microchannels. The first junction divides the second microchannel into a first branch and a second branch, wherein the first branch is between the first open end of the second microchannel and the first junction, and the second branch is between the first junction and the second open end of the second microchannel. The second microchannel is in fluid communication with the second inlet port and the third microchannel through the first and second open ends, respectively, thereby forming a second junction of the second and third microchannels. The second junction divides the third microchannel into a first branch and a second branch, wherein the first branch is between the first open end of the third microchannel and the first junction, and the second branch is between the first junction and the second open end of the third microchannel. The third microchannel is in fluid communication with the first and second outlet ports through the first and second open ends, respectively. In one embodiment, the liquid medium of interest is input to the first inlet port of the first particle separation units.

In one embodiment, each of the first and second particle separation units further has a hurdle protruded inwards from the first side wall of the second microchannel in the second branch. The hurdle has a cross-sectional geometric shape with a height, h. The cross-sectional geometric shape is selected from the group consisted of a triangle, a square, a rectangle, a semi-circle and a polygon. The height h is less than the width, w2, of the second microchannel so as to allow particles of the liquid medium of interest to pass through the second branch of the second microchannel. The hurdle is formed of a dielectric material.

The flow focusing unit has a first, second and third inlet ports, an outlet port, and a first and second microchannel, each of the first and second microchannels formed with a first open end, an opposite, second open end, and a first side wall and an opposite, second side wall defining a corresponding channel width therebetween. Each channel width is in a range of about 0.1-1,000 μm, preferable in a range of about 1-500 μm. The first microchannel is in fluid communication with the first inlet port and the outlet port through its first and second open ends, respectively. The second microchannel is in fluid communication with the second and third inlet ports through its first and second open ends, respectively. The first and second microchannels are in fluid communication with each other through a junction formed therein. The junction divides each of the first and second microchannels into a first branch and a second branch, wherein the first branch of each of the first and second microchannels is between the first open end of the corresponding microchannel and the junction, and the second branch of each of the first and second microchannels is between the junction and the second open end of the corresponding microchannel.

In one embodiment, the first particle separation unit, the second particle separation unit, and the flow focusing unit are adapted such that the first inlet port of the second particle separation unit coincides with one of the first and second outlet ports of the first particle separation unit, and the first inlet port of the flow focusing unit coincides with one of the first and second outlet ports of the second particle separation unit.

Furthermore, the flow cytometer comprises a fluid control member configured to control flow of the liquid medium in the microchannel structure. In one embodiment, the fluid control member includes a plurality of electrodes, each electrode placed in a corresponding port of the first and second particle separation units and the flow focusing unit; and a power source electrically coupled with the plurality of electrodes for individually applying voltages to each of the plurality of electrodes so as to generate desired electrokinetically microfluidic flows in the first and second particle separation units and the flow focusing unit for separating and transporting the particles in the liquid medium. The fluid control member further comprises a controller in communication with the power source and the plurality of electrodes for regulating voltages applied to each of the plurality of electrodes.

Moreover, the flow cytometer comprises a second substrate having a first surface and an opposite, second surface. The second substrate is bonded to the first substrate such that the first surface of the second substrate is substantially in contact with the second surface of the first substrate, thereby sealing the microchannel structure formed in the body portion of the first substrate. In one embodiment, each of the first and second substrates is formed of a corresponding dielectric material, wherein the first substrate is formed of polydimethylsiloxane (PDMS), and the second substrate is formed of glass, respectively.

In operation, the voltages are applied to the electrodes placed in the first and second inlet ports and the first and second outlet ports of each of the first and second particle separation units, respectively, such that the generated electrokinetically microfluidic flows cause (i) a liquid medium of interest introduced to the first inlet port and a buffer solution introduced to the second inlet port to move along the first microchannel and the first branch of the second microchannel, respectively, towards the first junction, and to merge into a stream of fluid therein; (ii) the merged stream of fluid to move along the second branch of the second microchannel towards and through the hurdle and towards the second junction, and to separate into a first and second streams of fluid therein; and (ii) the separated first and second streams of fluid to move along the first and second branches of the third microchannels towards the first and second outlet ports, respectively, of the corresponding particle separation unit, wherein the separated first stream of fluid contains particles that are substantially different from these contained in the separated second stream of fluid. In addition, the voltages are applied to the electrodes placed in the first, second and third inlet ports and the outlet ports of the flow focusing unit such that the generated electrokinetically microfluidic flows cause a particle-carrying flow from the first inlet port, a first buffer solution flow from the second inlet port, a second buffer solution flow from the third inlet port to move towards and meet at the junction, and to move towards the outlet port; and the first buffer solution flow and the second buffer solution flow to squeeze the particle-carrying flow to a desired size in the second branch of the first microchannel, thereby focusing the particle-carrying flow such that each particle moves singly along the second branch of the first microchannel towards the outlet port.

Additionally, the flow cytometer comprises an optical detection unit configured for counting and differentiating particles in the liquid medium. In one embodiment, the optical detection unit includes one or more input optical fibers. Each input optical fiber is positioned over the second branch of the first microchannel of the flow focusing unit from the first substrate for delivering a corresponding beam of laser thereto to illumine the particles in the focused stream of fluid passing therethrough. The optical detection unit also includes one or more output optical fibers. Each output optical fiber is positioned opposite to a corresponding input optical fiber from the second substrate such that when a particle passes through a position to which a beam of laser is delivered from the corresponding input optical fiber, the output optical fiber receives a signal associated with the particle. In one embodiment, each of the one or more input optical fibers and the one or more output optical fibers comprises a multimode optical fiber that has a diameter in a range of about 10-200 μm. The signal associated with the particle comprises a fluorescent signal emitted from the particle in response to the illumination of the beam of laser. The optical detection unit further includes a plurality of detectors coupled with the one or more output optical fibers for recording signals received from the one or more output optical fibers. The recorded signals are usable for counting and differentiating the particles passing through the second branch of the first microchannel of the flow focusing unit. Additionally, the optical detection unit may also include a plurality of filters. Each filter is coupled between the one or more output optical fibers and one of the plurality of detectors, respectively.

In another aspect, the present invention relates to a flow cytometer. In one embodiment, the flow cytometer comprises a microchannel structure adapted for transporting a fluid medium containing one or more types of particles; means for generating electrokinetically microfluidic flows to transport the fluid medium in the microchannel structure so as to differentiate the one or more types of particles in the fluid medium therein; and an optical detection system configured to detect the differentiated one or more types of particles of the fluid medium.

In one embodiment, the microchannel structure includes at least one particle separation unit, wherein the at least one particle separation unit comprises at least one inlet port, a first and second outlet forts, and at least one channel in fluid communication with the at least one inlet port and the first and second outlet ports, wherein the at least one microchannel is formed with at least one side wall and a hurdle protruded inwards from the at least one sidewall such that when the fluid medium is introduced into the at least one microchannel and passes through the hurdle, the one or more types of particles are dielectrophoretically differentiated into a first and second groups of particles in accordance with their sizes, wherein the first and second groups of particles move towards the first and second outlet ports, respectively. The hurdle has a cross-sectional geometric shape selected from the group consisted of a triangle, a square, a rectangle, a semi-circle and a polygon. The microchannel structure further comprises a flow focusing unit in fluid communication with the at least one particle separation unit, wherein the flow focusing unit comprises at least one inlet port, an outlet port and at least one microchannel in fluid communication with the at least one inlet port and the outlet port, and wherein when one of the first and second groups of particles received in a corresponding outlet port of the at least one particle separation unit is introduced to the at least one microchannel from the at least one input port, each particle moves singly along the at least one microchannel towards the outlet port.

In one embodiment, the optical detection system includes one or more input optical fibers, each input optical fiber positioned over the at least one microchannel of the flow focusing unit for delivering a corresponding beam of laser thereto to illumine the particles passing therethrough; one or more output optical fibers, each output optical fiber positioned opposite to a corresponding input optical fiber such that when a particle passes through a position to which a beam of laser is delivered from the corresponding input optical fiber, the output optical fiber receives a signal associated with the particle; and a plurality of detectors coupled with the one or more output optical fibers for recording signals received from the one or more output optical fibers, wherein the recorded signals are usable for counting and differentiating the particles passing through the second branch of the first microchannel of the flow focusing unit.

In one embodiment, the liquid medium of interest comprises a biological fluid of a living subject, wherein the biological fluid comprises blood or urine, and wherein the blood or urine comprises one or more types of particles or cells, wherein the one or more types of cells are differentiatable by their sizes, functions or a combination of them.

In yet another aspect, the present invention relate to a method for counting and differentiating particles in a liquid medium of interest, where the liquid medium of interest contains one or types of particles. In one embodiment, the method includes the steps of providing a microchannel structure on a first substrate; generating electrokinetically microfluidic flows to transport the liquid medium in the microchannel structure so as to differentiate the one or more types of particles in the liquid medium therein; and detecting the differentiated one or more types of particles in the liquid medium.

In one embodiment, the microchannel structure comprises at least one particle separation unit. The at least one particle separation unit comprises a first and second inlet ports, a first and second outlet ports, and a first to third microchannels, each of the first to third microchannels formed with a first open end, an opposite, second open end, and a first side wall and an opposite, second side wall defining a corresponding width therebetween. The first microchannel is in fluid communication with the first inlet port and the second microchannel through the first and second open ends, respectively, thereby forming a first junction that divides the second microchannel into a first branch and a second branch, wherein the first branch is between the first open end and the first junction, and the second branch is between the first junction and the second open end. The second microchannel is in fluid communication with the second inlet port and the third microchannel through its first and second open ends, respectively, thereby forming a second junction that divides the third microchannel into a first branch and a second branch, wherein the first branch is between the first open end and the second junction, and the second branch is between the second junction and the second open end. The third microchannel is in fluid communication with the first and second outlet ports through its first and second open ends, respectively. In one embodiment, the at least one particle separation units further has a hurdle protruded inwards from the first side wall of the second branch of the second microchannel. The hurdle has a cross-sectional geometric shape with a height, h, wherein the cross-sectional geometric shape is selected from the group consisted of a triangle, a square, a rectangle, a semi-circle and a polygon, and the height h is less than the width, w2, of the second microchannel so as to allow one or more types of particles of the liquid medium to pass through the second branch of the second microchannel.

The microchannel structure further comprises a flow focusing unit in fluid communication with the at least one particle separation unit, wherein the flow focusing unit further has a first, second and third inlet ports, an outlet port, and a first and second microchannels, each of the first and second microchannels formed with a first open end, an opposite, second open end, and a first side wall and an opposite, second side walls defining a width therebetween. The first microchannel is in fluid communication with the first inlet port and the outlet port through its first and second open ends, respectively. The second microchannel is in fluid communication with the second and third inlet ports through its first and second open ends, respectively. The first and second microchannels are in fluid communication with each other through a junction formed therein, the junction divides each of the first and second microchannels into a first branch and a second branch. The first branch of each of the first and second microchannels is between the first open end of the corresponding microchannel and the junction, and wherein the second branch of each of the first and second microchannels is between the junction and the second open end of the corresponding microchannel.

The step of generating electrokinetically microfluidic flows comprises the steps of placing an electrode into a corresponding port for each of the first and second inlet ports and the first and second outlet ports of the at least one particle separation unit and the first, second and third inlet ports and the outlet port of the flow focusing unit; and individually applying voltages to each of the placed electrodes to generate electrokinetically microfluidic flows in the at least one particle separation unit and the flow focusing unit.

The generated electrokinetically microfluidic flows in the at least one particle separation unit cause (1) a liquid medium of interest introduced to the first inlet port and a buffer solution introduced to the second inlet port to move along the first microchannel and the first branch of the second microchannel, respectively, towards the first junction, and to merge into a stream of fluid therein; (2) the merged stream of fluid to move along the second branch of the second microchannel towards and through the hurdle and towards the second junction, and to separate into a first and second streams of fluid therein; and (3) the separated first and second streams of fluid to move along the first and second branches of the third microchannels towards the first and second outlet ports, respectively, of the corresponding particle separation unit, wherein the separated first stream of fluid contains particles that are substantially different from these contained in the separated second stream of fluid.

Furthermore, the generated electrokinetically microfluidic flows in the flow focusing unit cause a particle-carrying flow from the first inlet port, a first buffer solution flow from the second inlet port, a second buffer solution flow from the third inlet port to move towards and meet at the junction, and to move towards the outlet port; and the first buffer solution flow and the second buffer solution flow to squeeze the particle-carrying flow to a desired size in the second branch of the first microchannel, thereby focusing the particle-carrying flow such that each particle moves singly along the second branch of the first microchannel towards the outlet port.

The detecting step comprises the steps of delivering at least one beam of laser to the second branch of the first microchannel of the flow focusing unit at a position to illumine a particle passing through the position; collecting signals for a period of time, each signal associated with a particle passing through the position; and analyzing the collected signals to determine the number and type of the particles passing through the second branch of the first microchannel of the flow focusing unit. In one embodiment, the signal associated with the particle comprises a fluorescent signal emitted from the particle in response to the illumination of the at least beam of laser.

The liquid medium of interest may comprise a biological fluid of a living subject. The biological fluid includes blood or urine. The blood or urine comprises one or more types of particles or cells. The one or more types of cells are differentiatable by their sizes, functions or a combination of them. The one or more types of cells may comprise CD4+ cells, and/or CD3+ cells. In one embodiment, the CD4+ cells and CD3+ cells are labeled with a first and second antibodies, respectively, where the first and second antibodies are excited with light of different wavelengths. The one or more types of cells are associated with a disease, which may be then detected and/or treated through the cells.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 shows schematically (a) an electrokinetically microfluidic flow cytometer lab-on-a-chip device and (b) a particle separation unit of the flow cytometer lab-on-a-chip device according to one embodiment of the present invention;

FIG. 2 shows schematically (a) a contour of a DC electric field around an insulating hurdle in a microchannel of a particle separation unit and (b) an enlarged view of a particle moving around the edge region of the insulating hurdle according to one embodiment of the present invention, where the darkness level indicates the magnitude of the DC electric field, the x-direction is alone the microchannel length (flow), and the y-direction is along the channel width, and the x-y coordinates are normalized by the channel width;

FIG. 3 shows schematically a vertical optical detection system according to one embodiment of the present invention;

FIG. 4 shows schematically a perspective view of a flow cytometer lab-on-a-chip device according to one embodiment of the present invention;

FIG. 5 shows superimposed sequential microscope images of the separation of polystyrene particles having sizes of about 6 μm and about 15 μm by an induced DC-DEP force according to one embodiment of the present invention;

FIG. 6 shows (a) schematically an electrokinetically controlled flow focusing system and (b) an image of a focused fluorescent particles stream according to one embodiment of the present invention, where the arrows indicate the flow directions;

FIG. 7 shows (a) partially optical fibers embedded in a PDMS chip, wherein the thinner fiber introduces the laser beam, the thicker fiber detects the laser, and a particle is detected once it passes through the laser beam, and (b) the detected optical signal strength, where each peak represents one particle;

FIG. 8 shows a comparison of CD4 and CD8 percentages from whole blood staining vs. ficoll-isolated PBMC, where whole blood was stained with antibodies, followed by lysis of RBC, and then run directly without washes, and subjects 1, 2, and 3 are HIV-uninfected, subjects 4 and 5 are HIV-infected, note lower CD4+ T cell percentages;

FIG. 9 shows representative flow cytometry plots of whole blood stained with CD3 (APC) and CD4 (FITC) antibodies, (a) forward and side scatter differentiates lymphocytes, monocytes, and Polymorphonuclear cells (PMNs), where horizontal lines represent relative size demarcations (based on forward scatter) that preferentially include lymphocytes (approximately 4-10 micron size), (b): gate on CD3+ cells (T cells), and (c) dots 910 represent CD3+ CD4+ T cells, note monocytes 920, very few of which are in this size gate, which whey stain dimly with anti-CD4 and do not express CD3; and

FIG. 10 shows the use of “TruCount” beads to evaluate absolute T cell numbers, (a) CD45, a marker for all white blood cells, vs. side scatter, PMNs 1010; monocytes 1020; CD3+ lymphocytes 1030; CD3+ CD4+ lymphoctes 1040, where size beads are at upper left, and calibration (“TruCount”) beads for counting are bright green 1050 at far right, and (b) CD3 and CD4 expression on CD45+ lymphocytes. Since the number of beads per tube and the volume of added blood are known, the absolute CD4+ T cell count can be calculated.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which has no influence on the scope of the invention. Additionally, some terms used in this specification are more specifically defined below.

### Definitions

The terms used in this specification generally have their ordinary meanings in the art, within the context of the invention, and in the specific context where each term is used.

Certain terms that are used to describe the invention are discussed below, or elsewhere in the specification, to provide additional guidance to the practitioner in describing the apparatus and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification. Furthermore, subtitles may be used to help a reader of the specification to read through the specification, which the usage of subtitles, however, has no influence on the scope of the invention.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “lab-on-a-chip” or its acronym “LOC”, as used herein, refers to a device that has at least one microchannel structure, and that integrates multiple laboratory functions (processes) on a single chip of only millimeters to a few square centimeters in size. The LOC is capable of handling substantially small fluid volumes down to less than picoliters to perform desired biological and/or chemical analysis.

As used herein, the term “microchannel” refers to a channel structure having a cross-sectional dimension, e.g., a width, a depth or a diameter, in a microscale range from about 0.1 μm to about 1 mm. According to the present invention, the microchannels preferably have a cross-sectional dimension between about 0.1 μm and 500 μm, more preferably between about 0.1 μm and 300 μm. A device referred to as being microscale includes at least one structural element or feature having such a dimension.

As used herein, the term “microfluidics” refers to the science of designing, manufacturing, and formulating devices and processes that deal with volumes of fluid on the order of nanoliters (nl) or picoliters (pl). A microfluidic device has one or more channels with a cross-sectional dimension less than 1 mm. Common fluids used in microfluidic devices include whole blood samples, bacterial cell suspensions, protein or antibody solutions and various buffers. Applications for microfluidic devices include, but not limited to, capillary electrophoresis, isoelectric focusing, immunoassays, flow cytometry, sample injection of proteins for analysis via mass spectrometry, PCR (polymerase chain reaction) amplification, DNA (deoxyribonucleic acid) analysis, cell manipulation, cell separation, cell patterning and chemical gradient formation. Many of these applications have utility for clinical diagnostics.

As used herein, the term “electrokinetics” refers to the science of electrical charges in moving substances, such as water or blood, which studies particle motion that is the direct result of applied electric fields. Electrokinetics includes electroosmosis, electrophoresis, dielectrophoresis and electrorotation.

Electroosmosis, also called electroendosmosis, is the motion of polar liquid through a membrane or other porous structure (generally, along charged surfaces of any shape and also through non-macroporous materials which have ionic sites and allow for water uptake, the latter sometimes referred to as “chemical porosity”) under the influence of an applied electric field.

When a solid surface is in contact with an aqueous solution, electrostatic charge will be established at the surface. These surface charges in turn attract the counter ions in the liquid to the region close to the solid-liquid interface to form the electrical double layer. In the electrical double layer region, there are excess counter ions (net charge). If the solid surface is negatively charged, the counter ions are the positive ions. Such an electrical double layer field is responsible for two basic electrokinetic phenomena: electroosmosis and electrophoresis. When an external electrical field is applied tangentially to the solid surface, the excess counter ions will move under the influence of the applied electrical field, pulling the liquid with them and resulting in electroosmotic flow. The liquid movement is carried through to the rest of the liquid in the microchannel by the viscous effect. In most LOC applications, electroosmotic flow is preferred over pressure driven flow. One of the reasons is the plug-like velocity profile of electroosmotic flow. This means that fluid samples can be transported without dispersion caused by flow shear. Furthermore, pumping a liquid through a small microchannel requires applying a very large pressure difference depending on the flow rate. This is often impossible because of the limitations of the size and the mechanical strength of the microfluidic devices. Electroosmotic flow can generate the required flow rate in very small microchannels without any applied pressure difference cross the channel. Additionally, using electroosmotic flow to transport liquids in complicated microchannel networks does not require any external mechanical pump or moving parts, it can be easily realized by controlling the applied electrical fields via electrodes.

Electrophoresis is the motion of a charged particle relative to the surrounding liquid under an applied electrical field. In a microchannel, the net velocity of a charged particle is determined by the electroosmotic velocity of the liquid and the electrophoretic velocity of the particle. If the surface charge of the particle is not strong or the ionic concentration of the liquid (e.g., typical buffer solutions) is high, the particle will move with the liquid. Using electrical fields to manipulate and transport particles and biological cells in microchannels is particularly suitable for LOC applications.

It should be noted that the applied electrical field has negligible effects on the cells, other than generating the flow and the cell motion. This can be appreciated by comparing the applied electrical field strength with the electrical field strength of the cells' electrical double layer (EDL) field, i.e., the field around each cell generated by the natural surface electrostatic charge. The typical EDL field strength is 100 mV/10 nm=100,000 V/cm, while the applied electrical field ranges from 10 V/cm to 100 V/cm.

Dielectrophoresis or its acronym “DEP” refers to a phenomenon in which a force is exerted on a dielectric particle when it is subjected to a non-uniform electric field. This force does not require the particle to be charged. All particles exhibit dielectrophoretic activity in the presence of electric fields. However, the strength of the force depends strongly on the medium and particles' electrical properties, on the particles' shape and size, as well as on the frequency of the electric field. Consequently, fields of a particular frequency can manipulate particles with great selectivity. This has allowed, for example, the separation of cells or the orientation and manipulation of nanoparticles.

### Overview of the Invention

The widespread use of benchtop flow cytometers has been limited by their size, cost, and unease-to-use. Therefore, a simple and compact flow cytometer would gain great relevance in biomedical and chemical applications and related fields. The present invention, among other things, provides an electrokinetically microfluidic flow cytometer LOC device that integrates multiple laboratory functions and/or processes on a single, small sized chip. By detecting different fluorescent signals carried by the cells, the flow cytometer LOC device can count and differentiate the cells. The electrokinetically microfluidic flow cytometer LOC device, among other things, possesses unique features absent in the conventional benchtop flow cytometers. These unique features include, but not limited to, (1) using substantially small amount of a sample, (2) performing all processes on a single chip, where all processes are fully electrokinetically controlled, with no mechanical moving parts, no tubing and valves, (3) fully automatic operation, (4) using electrokinetic microfluidic means such as DC-DEP to separate small sized components (e.g., lysed red blood cells, platelets, proteins, etc) and very large sized components (e.g., monocytes) before these cells are counted and detected, which significantly minimizes the possible false positive due to the non-specific adsorption of the fluorescent dyes to these components, (5) miniaturization of a laser-optical fiber based photo detection system that eliminates false positive signals produced by the non-specific adsorption of the fluorescent dyes to some undesired cells (e.g., monocytes), thereby, significantly increasing the accuracy and reliability of the cell counting and detection, (6) completing flow cytometry analysis in substantially shorter time (within a few minutes), in comparison with several hours as required by the conventional benchtop flow cytometors, and (7) portability. The flow cytometor LOC device is a fully automatic, stand-alone, and portable device. The flow cytometor LOC device has wide applications in biomedical diagnosis of infectious diseases (e.g., HIV), cancers (e.g., leukemia), and other diseases that can be diagnosed by analyzing cells in blood and in body fluids. The flow cytometor LOC device is particularly useful in field applications or point-of-testing applications where only very small amount of samples are available and immediate diagnostics is required (e.g., diagnostics of HIV and leukemia).

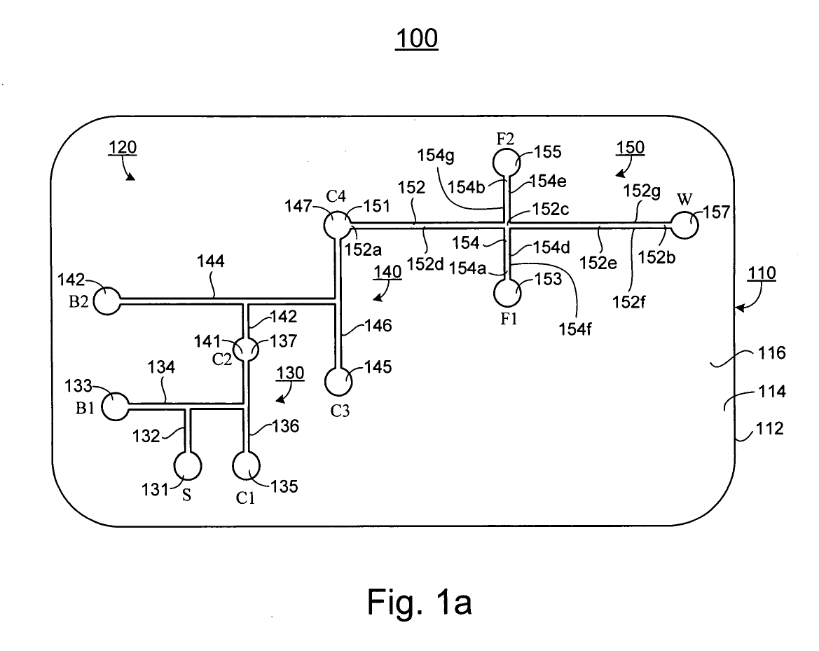

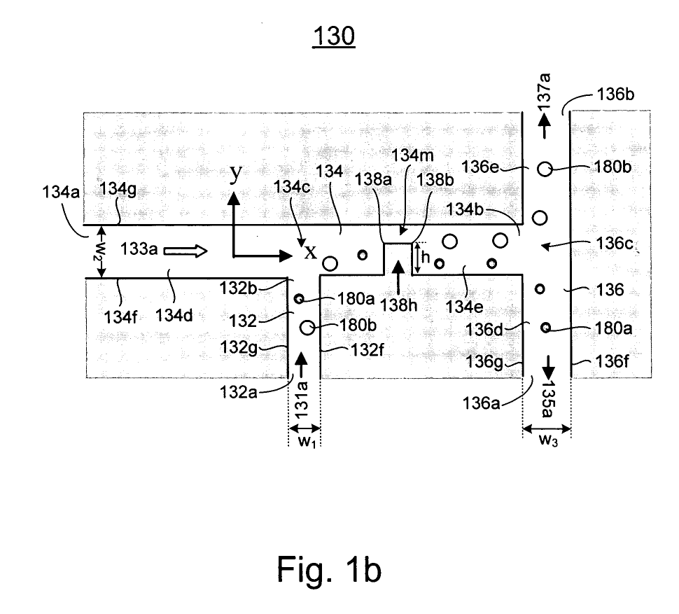

The description of the electrokinetically microfluidic flow cytometer LOC device in connection with other unique features of the present invention will be made as to the embodiments of the present invention in conjunction with the accompanying drawings in FIGS. 1-10.

Referring to FIG. 1, an electrokinetically microfluidic flow cytometer 100 is schematically shown according to one embodiment to the present invention, which is an LOC device. The flow cytometer 100 is adapted for counting and differentiating particles in a liquid medium of interest. For example, the liquid medium of interest can be a biological fluid of a living subject, such as blood or urine. The blood or urine has one or more types of particles or cells. The one or more types of cells are differentiatable by their sizes, functions or a combination of them. The change or percentage of the one or more types of cells in the blood or urine may be associated with a disease.

The flow cytometer 100 includes a first substrate 110 having a first surface 112 and an opposite, second surface 114 defining a body portion 116 therebetween, and a microchannel structure 120 that is formed in the body portion 116 of the first substrate 110. The microchannel structure 120 includes a first particle separation unit 130, a second particle separation unit 140, and a flow focusing unit 150.

The first and second particle separation units 130 and 140 are structurally and functional similar to each other, as shown in FIG. 1 a. Each of the first and second particle separation units 130 (140) has a first and second inlet ports (wells) 131 (141) and 133 (143), a first and second outlet ports 135 (145) and 137 (147), and a first to third microchannels 132 (142), 134 (144) and 136 (146). Without intent to limit the scope of the invention, only the first particle separation units 130 according to the embodiment of the present invention is illustrated and described in further details as follows.

As shown in FIGS. 1a and 1b, and particularly in FIG. 1b, in the first particle separation units 130, each of the first, second and third microchannels 132, 134 or 136 is formed with a first open end 132a, 134a or 136a, an opposite, second open end 132b, 134b or 136b, with a first side wall 132f, 134f or 136f and an opposite, second side wall 132g, 134g or 136g defining a corresponding channel width, w1, w2 or w3, therebetween, respectively. Each microchannel 132, 134 or 136 has at least one cross-sectional dimension in a microscale. The first, second and third microchannels 132, 134 or 136 can have the same cross-sectional dimension or substantially different cross-sectional dimensions. In one embodiment, each channel width, w1, w2 or w3, is in a range of about 0.1-1,000 μm, preferable in a range of about 1-500 μm.

The first microchannel 132 is in fluid communication with the first inlet port 131 and the second microchannel 134 through the first and second open ends 132a and 132b, respectively, thereby forming a first junction 134c of the first and second microchannels 132 and 134. The first junction 134c is a T-like junction that divides the second microchannel 134 into a first branch 134d and a second branch 134e. The first branch 134d is between the first open end 134a of the second microchannel 134 and the first junction 134c, and the second branch 134e is between the first junction 134c and the second open end 134b of the second microchannel 134. The second microchannel 134 is in fluid communication with the second inlet port 133 and the third microchannel 136 through the first and second open ends 134a and 134b, respectively, thereby forming a second junction 136c of the second and third microchannels 134 and 136. The second junction 136c is a T-like junction that divides the third microchannel 136 into a first branch 136d and a second branch 136e. The first branch 136d is between the first open end 136a of the third microchannel 136 and the first junction 136c, and the second branch 134e is between the first junction 134c and the second open end 134b of the third microchannel 136. The third microchannel 136 is in fluid communication with the first and second outlet ports 135 and 137 through the first and second open ends 136a and 136b, respectively.

The first particle separation units 130 further has a hurdle 138 protruded inwards from the first side wall 134f of the second branch 134e of the second microchannel 134. The hurdle 138 has a cross-sectional geometric shape of rectangle with a height, h. The cross-sectional geometric shape can also be a triangle, a square, a semi-circle or a polygon. The height h of the hurdle 138 is less than the width, w2, of the second microchannel 134, thereby allowing particles of the liquid medium of interest to pass through the second branch 134e of the second microchannel 134. The hurdle 138 is formed of a dielectric material.

According to one embodiment of the present invention, the separation of particles in the liquid medium of interest is performed by a DC-dielectrophoresis (DEP) force. Consider a suspension of dielectric particles in a dielectric fluid. In the presence of an applied electric field, the particle and the surrounding medium are electrically polarized and the surface charge accumulates at the interfaces due to the difference in electric properties. The distribution of the surface charge of the particle gives rise to an induced dipole moment. A dipole tends to align in parallel with the local electric field. In a non-uniform electric field, the forces acting on the opposite charges of a dipole become asymmetric. As a result, there exists a non-zero net force, called DEP force, acting on the particle. The induced motion of the particle due to the DEP force is known as dielectrophoresis [10].

For a non-conducting and electrically neutral particle under a DC field, an approximate expression of the DEP force is given by

F_{DEP}=−2πε_{f}a³(E·∇)E, (1)

where ε_{f} is the liquid dielectric constant and a is the particle radius, E is the local electric field. It has been shown that this equation is valid for biological cells of spherical-shell structure [11]. The negative sign means that the DEP force always directs to the region of the lower electric-field strength, i.e., negative DEP.

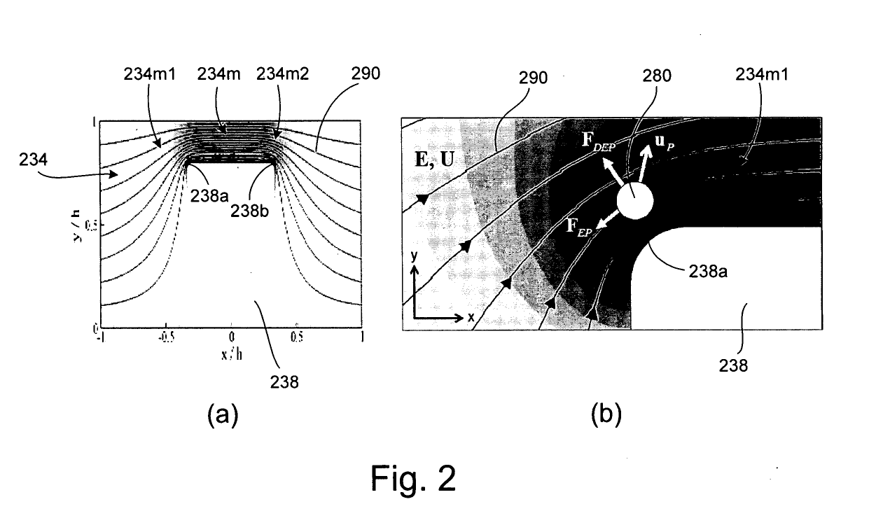

Referring now to FIG. 2, a non-uniform local electric field 290 at the area of a hurdle 230 in a fluidic microchannel 234 and an induced DEP force, F_{DEP}, on a particle 280 moving along the electric filed 290 are schematically shown. The non-uniform local electric field 290 at the hurdle 238 is corresponding to an applied DC field disturbed by the hurdle 238. The hurdle 238 is attached on or protruded from one side of the microchannel to form an abruptly narrow section 234m in the microchannel 234. Since only the liquid (an aqueous solution) conducts the electrical field, the narrow section 234m of the microchannel 234 generates a spatially non-uniform DC electrical field 290 in the liquid near the hurdle 238. An enlarged view of the local electrical field 290 near the up-stream corner 238a of the hurdle 238 is shown in FIG. 2b. Under the combined effect of the electroosmotic flow (EOF) and the electrophoresis (EP), a particle 280 moves towards the entrance region 234m1 of the narrow section 234m of the microchannel 234. As shown in FIG. 2b, the electric field 290 is stronger in the region close to a corner 238a of the hurdle 238 than that in the region far from to the corner 238a of the hurdle 238. Since the negative DEP force, FDEP, directs to the region of lower electric-field strength, the particle 280 experiences a repulsive force from the corner 238a of the hurdle 238. The magnitude of the repulsive DEP force is proportional to the volume of the particle 238 and the local value of (E·∇)E, as indicated by equation (1).

For example, the repulsive DEP force on a 15 μm particle is 27 times of that on a 5 μm particle under the same conditions. Therefore, a larger particle is subject to a stronger DEP force and tends to be pushed further away from the corner compared with a smaller particle. The similar DEP repulsion occurs when the particle passes by the other corner 238b of the hurdle 238. As a result, the trajectory shift (in y-direction) is different for particles of different sizes and hence particles are separatable by size.

Referring back to FIG. 1b, a particle 180a is smaller than a particle 180b in a liquid medium of interest. For the particle separation units 130, DC electrical fields are applied to four electrodes placed in the first and second inlet ports (wells) and the first and second outlet ports (wells). A hurdle 138 is formed on one side wall 134f of the microchannel 134 to form an abruptly narrow section 134m. Since only the liquid medium of interest conducts the electrical field, the narrow section 134m of the microchannel 134 generates a spatially non-uniform DC electrical field in the liquid medium near the hurdle 138. The liquid medium having a mixture of large and small cells 180b and 180a is introduced into the particle separation unit 130 from the first microchannel 132. As explained above, the negative DC-DEP force at the corners 138a and 138b of the hurdle 138 pushes the larger cells 180b further from the corner 138b of the hurdle 138 than the smaller cells 180a is, and thus generates different trajectories for smaller and larger cells 180a and 180b once they pass the hurdle 138. After passing through the hurdle 138, a T-shaped channel structure 136c is used so that the separated small cells 180a and the separated large cells 180b are drawn into the first outlet port (well) 136a and the second outlet port (well) 136b, respectively, by electrokinetically microfluidic flows.

To efficiently separate particles or blood cells using the flow cytometer LOC device, the design parameters, e.g., the hurdle size and position and the controlling parameters, e.g., applied voltages, of the flow cytometer LOC device need to be optimized. This can be done theoretically and experimentally. Theoretically, the influences of different parameters on the particle (cell) trajectory are simulated using a complicated theoretical model and numerical simulation [12, 15-26], so as to obtain the optimal design parameters and controlling parameters.

Experimentally, fixed human blood cells are used. In addition, fluorescent (carboxylate-modified) polystyrene particles of different sizes: 1 μm, 3 μm, 4 μm, 6 μm, 10 μm, 12 μm, and 15 μm in diameter (Bangs Laboratory Inc.) are used as sample particles for evaluation. These particle sizes are similar to the size of typical blood cells and the small components involved in the samples.

At first, all the microchannels 132, 134 and 136 and all the wells (inlet and outlet ports) 131, 133, 135 and 137 are primed with about 1 mM sodium carbonate buffer solution. Then, the cells or particle mixture (a liquid medium of interest) and a buffer solution are introduced into the first and second inlet ports (wells) 131 and 133 with a syringe. Other tools can also be used to practice the present invention. A high-voltage DC power supply (Labsmith HVS448) is used to supply voltages to the four platinum electrodes submerged in the wells 131, 133, 135 and 137 as so to generate desired electrokinetically microfluidic flows to drive the liquid medium through the particle separation unit 130. A voltage controller coupled with the high-voltage DC power supply (not shown) is used to adjust independently the voltage applied to each of the four electrodes. Following the results of the numerical simulations as the guidance, the voltages applied to the four electrodes are adjusted such that in operation, the liquid medium of interest introduced into the first inlet port 131 and the buffer solution introduced into the second inlet port 133 move along the first microchannel 132 and the first branch 134d of the second microchannel 134, respectively, towards the first junction 134c, and merge into a stream of fluid therein. The merged stream of fluid then moves along the second branch 134e of the second microchannel 134 towards the hurdle 138 and passes through the hurdle 138. As described above, an induced DC-DEP force at the corners 138a and 138b of the hurdle 138 pushes the larger cells 180b in the liquid medium further from the corner 138b of the hurdle 138 than the smaller cells 180a in the liquid medium are, thereby, separating the cells into two groups according to the cell sizes. The group of the separated cells in small sizes and the group of the separated cells in large sizes move along the first and second branches 136d and 136e of the third microchannels 136 towards the first and second outlet ports (wells) 135 and 137, respectively.

The whole separation process is completed within about 60 seconds, and the EOF flow rate in the microchannels is small, the effect of the pressure-driven flow is minimized by using sufficiently large well size and by carefully balancing the liquid level in four wells. The cell/particle motion is monitored by a fluorescent microscope (MBA801, Nikon, Inc., Japan) and recorded by a progressive CCD camera (QImaging, Inc., British Columbia, Canada).

As shown in FIG. 1a, the flow focusing unit 150 has a first, second and third inlet ports 151, 153 and 155, an outlet port 157, and a first and second microchannel 152 and 154. Each of the first and second microchannels 152 (154) is formed with a first open end 152a (154a), an opposite, second open end 152b (154b), a first side wall 152f (154f) and an opposite, second side wall 152g (154g) defining a corresponding channel width therebetween. Each channel width is in a range of about 0.1-1,000 μm, preferable in a range of about 1-500 μm. In this embodiment, the first microchannel 152 is in fluid communication with the first inlet port 151 and the outlet port 157 through its first and second open ends 152a and 152b, respectively. The second microchannel 154 is in fluid communication with the second and third inlet ports 153 and 155 through its first and second open ends 154a and 154b, respectively. The first and second microchannels 152 and 154 are in fluid communication with each other through a junction 152c formed therein. As shown in FIG. 1a, the junction 152c divides each of the first and second microchannels 152 and 154 into a first branch 152d (154d) and a second branch 152e (154e). The first branch of each of the first and second microchannels 152 and 154 is between the first open end of the corresponding microchannel 152 and 154 and the junction, and the second branch of each of the first and second microchannels 152 and 154 is between the junction and the second open end of the corresponding microchannel 152 and 154.

In one embodiment, each of the first, second and third inlet ports 151, 153 and 155, and the outlet port 157 is provided with a corresponding electrode (not shown). These electrodes are electrically coupled with a high-voltage DC power supply (not shown) and a voltage controller (not shown) for applying voltages thereto to generate electrokinetically microfluidic flows in the flow focusing unit 150. The voltages are applied such that the generated electrokinetically microfluidic flows cause a corresponding group of the separated cells in the first inlet port 151 and the buffer solution introduced to the second and third inlet ports 153 and 155 to move towards the junction 152c, to meet at the junction 152c, and to move towards the outlet port 157 along the second branch 152e of the first microchannel 152. Because the flows of the corresponding group of the separated cells from the first inlet port 151 and the buffer solution from the second and third inlet ports 153 and 155 are laminar flows and do not mix when they move along the second branch 152e of the first microchannel 152, 654d and 654e. By adjusting the flow rates, i.e., adjusting the electrical potentials, the two side flows (buffer solution) squeeze the central cell-carrying flow to a desired size, thereby focusing the corresponding group of the separated cells in the second branch 152e of the first microchannel 152. In this case, particles (cells) 680a and 680b singly pass through the detecting point.

The merged stream of fluid is focused by the electrokinetically microfluidic flows moving towards the junction 152c, from the first branch and second branch 153d and 153e of the second microchannel 153, such that each particle in the merged stream of fluid moves singly along the second branch 152e of the first microchannel 152 towards the outlet port 157.

In this embodiment shown in FIG. 1a, the first inlet port 141 of the second particle separation unit 140 coincides with one of the first and second outlet ports 135 and 137 of the first particle separation unit 130, and the first inlet port 151 of the flow focusing unit 150 coincides with one of the first and second outlet ports 145 and 147 of the second particle separation unit 140, such that the first particle separation unit 130, the second particle separation unit 140, and the flow focusing unit 150 are in fluid communication with each another.

In one embodiment, the flow cytometer 100 may have a second substrate having a first surface and an opposite, second surface. The second substrate is bonded to the first substrate 110 such that the first surface of the second substrate is substantially in contact with the second surface of the first substrate, thereby sealing the microchannel structure 120 formed in the body portion 116 of the first substrate 110. In one embodiment, each of the first and second substrates is formed of a corresponding dielectric material, wherein the first substrate is formed of polydimethylsiloxane (PDMS), and the second substrate is formed of glass, respectively. The microchannel structure 120 in the PDMS substrate, in one embodiment, is fabricated following the soft lithography protocol [13]. A detailed fabrication procedure is described in reference [14].

As disclosed above, the microfluidic flow cytometer of the present invention, among other things, includes a microchannel structure having several distinctive functional units: a first DC-DEP separation unit, a second DC-DEP separation unit, and a flow focusing unit. These units are operated in a time sequence. Since all microchannels are connected and there are no mechanical valves, it is critical to control the flow of liquid in the microchannel network structure, i.e., control the flow directions in certain microchannels while keeping liquid in other channels stationary. This is realized by controlling the applied electrical field, i.e., different voltages at different electrodes in different wells (ports). Such an ability to control the electrokinetically microfluidic flow in inter-connected microchannels play a crucial role in the development of fully automatic microfluidics LOC devices that require multiple-steps, sequential reagents/solutions delivery/washing and flow switching [17, 28-32]. According to the present invention, the automatic, electrokinetically microfluidic flow is controlled, not only with spatial precision but also with temporal precision. These flow controls include the flow direction, flow switching and reagent holding in the wells (reservoirs).

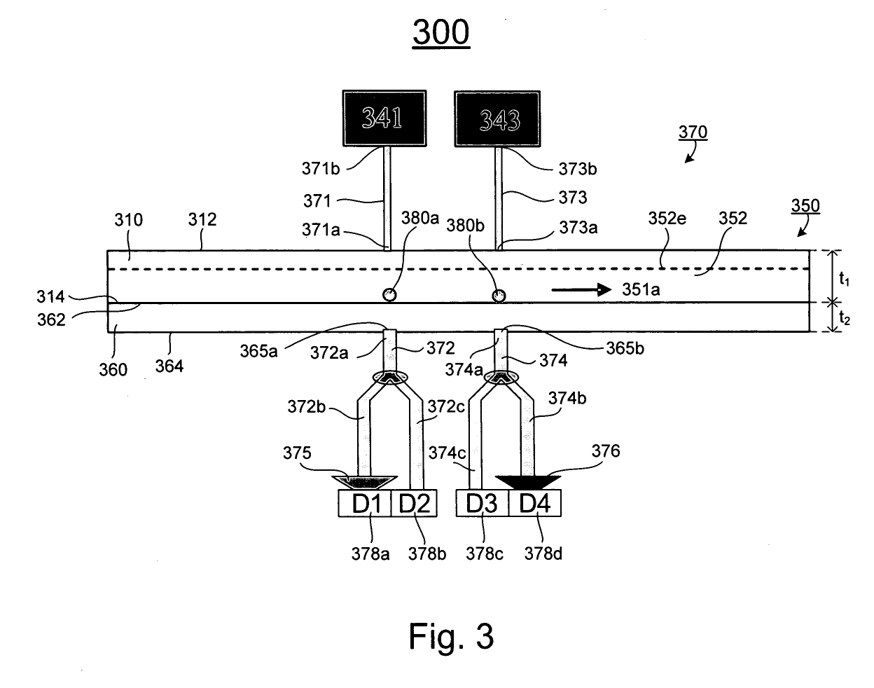

Referring to FIG. 3, a flow cytometer 300 according to one embodiment of the present invention also includes an optical detection unit 370 for counting and differentiating the particles in the liquid medium. In the embodiment, a vertical detection method is employed, which reduces the complexity of making the lab-on-a-chip (device) and the cost, and thus makes the lab-on-a-chip disposable.

The optical detection unit 370 includes one or more input optical fibers. In the embodiment shown in FIG. 3, two optical fibers 371 and 373 are utilized to practice the present invention. Each optical fiber 371 (273) has a first end 371a (373a) and an opposite, second end 371b (373b) coupled to two lasers 341 and 342, respectively. In one embodiment, two 100 μm fiber-coupled lasers, one emits light in red (650 nm) and the other in blue (488 nm). They are small, simple and inexpensive. The red laser used to practice the present invention is made from Lasermate Group, Inc., California. Other types of lasers can also be used to practice the present invention. Additionally, the first end of each optical fiber 371 (273) is positioned over the second branch 352e of the first microchannel 352 of the flow focusing unit 350 from the first substrate 310 for delivering a corresponding beam of laser thereto to illumine the particles, for example, 380a and 380b, in the focused stream of fluid 351a when they pass the positions underneath the two optical fibers 371 and 373.

The optical detection unit 370 also includes one or more output optical fibers. As shown in FIG. 3, two optical fibers 372 and 374 are employed in the embodiment of the present invention, each optical fiber 372 (374) having a working end 372a (374a). The working end 372a (374a) of each optical fiber 372 (274) is positioned opposite to a corresponding input optical fiber 371 (273) from the second substrate 360 such that when a particle (cell) 380a (380b) passes through a position to which a beam of laser is delivered from the corresponding input optical fiber 371 (373), the output optical fiber 372 (374) receives a signal associated with the particle (cell) 380a (380b). The signal associated with the particle (cell) 380a (380b) comprises a fluorescent signal emitted from the particle in response to the illumination of the beam of laser. In one embodiment, each of the one or more input optical fibers and the one or more output optical fibers comprises a multimode optical fiber that has a diameter in a range of about 10-200 μm.

The optical detection unit 370 further includes detectors 378a-378d coupled with the two optical fibers 372 and 374 for recording signals received from the one or more output optical fibers 372 and 374. After electronic amplification, each recorded signal is fed to the data acquisition card inside a computer for processing as so to count and differentiate the particles passing through the second branch 352e of the first microchannel 352 of the flow focusing unit 350.

For detecting the fluorescent emission from the cells 380a and 380b, a FITC filter 375 is used for the blue laser 341, while a Cy5 filter 376 is used for the red laser 342. Additionally, a silicon photodiode array (Hamamatsu, USA) is also employed. The Si photodiode array includes 10 Si PIN photo-detectors and each of them is coupled with a fiber of 100 μm in diameter.

Since the cells 380a (380b) are slightly heavier than water, they move along a bottom channel wall 314. The top layer (substrate) 310 of the LOC device is a thin PDMA plate 310 having a thickness of t₁ defined between its first surface 312 and its opposite, second surface 314, and the bottom layer (substrate) 360 is a thin glass plate having thickness of t₂=150 μm, which is defined between its first surface 362 and its opposite, second surface 364. The detection microchannel is 100 μm in width and 50 μm in depth. As illustrated in FIG. 3, the output sensing (photo-detecting) fibers 372 and 374 approach the microchannel 352 and the cells 380a and 380b from the bottom surface 364 of the glass substrate 360. The excitation lights are introduced by optical fibers from the top surface 312 of the PDMA plate 310. The fiber ends 371a, 373a and 372a, 374a touch the bottom glass plate 360 and the top PDMS plate 310, respectively. Additionally, a fiber positioner is adapted for holding and aligning the fibers 371-374 with the fluidic channel 352. In one embodiment, refractive index matching oil is applied between fiber ends 371 a, 373a and 372a, 374a and the top surfaces 312 of the PDMA plate 310 and the bottom surface 364 of the glass plate 360 to reduce both excitation power and fluorescent emission loss.

In order to make the cell counting more accurate and reliable, four photo detectors D1-D4 378a-378d are deployed at two locations 365a and 365b opposite to the two excitation laser beams delivered by the fibers 371 and 373, as shown in FIG. 3. At each location 365a (365b), a specific excitation laser 341 (343) is introduced from the top surface 312 of the LOC device to the liquid medium (sample) 351a through an optical fiber 371 (373) and an optical fiber 372 (374) underneath the LOC device collects the light signal emitted from the cell 380a (380b) responsive of the excitation of the corresponding laser. The collected light signal is split into two branches 372b (374b) and 372c (374c). One 372c (374c) goes directly into the photo diode detector D2 378b (D3 378c) and the other 372b (374b) goes through a filter 375 (376) first and then reaches another photo diode detector D1 378a (D4 378c). The filter 375 (376) is adapted for passing the specific emission wavelength for the specific dye tagged on CD4+ or CD3+ cells. In one embodiment, CD4+ cells are labeled with AlexaFluor-488-conjugated antibodies (only excited with the 488 nm wavelength laser) and CD3+ cells are labeled with AlexaFluor-647-conjugated antibodies (only excited with the 650 nm wavelength laser) (Becton Dickenson, San Jose, Calif.). The peak emission wavelength is 665 nm for AlexaFluo-647, and 520 nm for AlexaFluo-488. Since the emission spectra of these fluorochromes do not overlap, compensation of the detector system is not necessary. In this embodiment, the small and portable optical detection system 370 is capable of detecting these two emission wavelengths.

In one embodiment, a Cy5 filter 376 is used to detect the AlexaFluor-647 emission, and a FITC filter 375 is used to detect AlexaFluor-488 emission. After the filter 375 (376), only the signal emitted from the individual cell 380a (380b) excited by the laser can be detected at the photo detector. Therefore, the signals collected by the photo detector D1 378a and the photo detector D4 378d in FIG. 3 can be used to determine the number of CD4 and CD3 cells, respectively. When a cell passes through a laser beam, it blockes the light path and generate a signal. The signal collected at the photo detector D2 (D3) without going through the optical filter indicates whether there is a cell passing the laser beam. Therefore, the signals collected at the photo detector D2 (D3) shows the total number of cells passing through the system.

While the fluorochromes are selected to have different excitation wavelengths and non-overlapping emission wavelengths, cells can have a low level of expression of markers that can confound their discrimination. For example, while small granulocytes can overlap in size with larger lymphocytes, they are CD3(−) and CD4(−) and readily differentiated from T lymphocytes. Monocytes can overlap lymphocytes by size, and have a low level CD4 expression, but are CD3 (−). Therefore, it is necessary to distinguish the false signals detected at photo diode detectors D1 and D4 that are generated by monocytes. Thus, a cell that shows a detectable AlexaFluor-488 emission (CD4) but weak or absent AlexaFluor-647 (CD3) emission would be considered as a monocyte. However, it should be determined whether a relatively strong AlexaFluor-488 emission signal detected at the photo detector D1 and a weak AlexaFluo-647 emission signal detected at the photo detector D4 are from the same cell.

The signals collected at the photo diode detector D3 enable one to distinguish this kind of false signal by comparing the signals collected at the four photo detectors. In one embodiment, all the signals collected at the four detectors D1-D4 are recorded with a timer in a microprocessor chip in the flow cytometer. Since the signals detected at the detectors D1 and D2 are from the same physical position, the signals simultaneously detected by D1 and D2 are from the same cell; D1 counts CD4 cells, while D2 counts events. Similarly the signals simultaneously detected by D3 and D4 are from the same cell; D3 counts events, while D4 counts CD3 cells. By cross-linking the events detected at D2 and D3, we can identify if a relatively strong AlexaFluo-488 emission signal detected at the photo detector D1 and a weak AlexaFluo-647 emission signal detected at the photo detector D4 are from the same cell.

To optimize the discrimination between CD3(+)CD4(+) T cells and CD3(−)CD4(+) monocytes, cell subsets are sorted to high purity with the FACSaria sorter, and the sorted subpopulations are precisely counted with the GUAVA counter. The GUAVA counter is specifically adapted to provide accurate cell counts of cells in suspension, and is used in cell processing laboratories to minimize variation from manual cell counting. In one embodiment, all isolated cells that is analyzed by the described methods is first quantified by the GUAVA. The purified cell subsets is mixed at defined ratios and simultaneously evaluated by the flow cytometer LOC device of the present invention and the FACSaria. As additional controls, the same preparation of cells with known numbers of CD3(+)CD4(+) T cells and CD3(−)CD4(+) monocytes are stained with a combination of anti-CD3-Alexa-488 and anti-CD14-Alexa 647 to specifically stain T cells vs. monocytes respectively. Likewise, several paired combinations of stains are used to internally validate results. These include additional antibodies specific for CD 19 (B lymphocytes) CD 16 and CD56 (NK cells) (both B cells and NK cells are CD3 negative) or CD45 (all white blood cells).

In operation, a control device according to the present invention is utilized to control the multiple steps of electrokinetic microfluidic processes, synchronize the microfluidics and optical detection, and collecting data and computing the results. In one embodiment, the control device may have at least four (4) analogue inputs, twelve (12) digital outputs, and one timer. Using the signal from the digital output, the voltages applied at different wells are controlled to achieve the desired flows at the different functional units in the flow cytometer lab-on-a-chip. As for the detection of the fluorescent signal, outputs of the four photodiode detectors are collected through the four analogue input channels continuously with the time references. To maximize the ratio of signal to noise of the optical signal detection, a lock-in amplification technique is used in the control device. The information collected at the four detectors is further analyzed to provide complete information (the total number and the percentage) of the CD4 and CD3 cells in the sample.

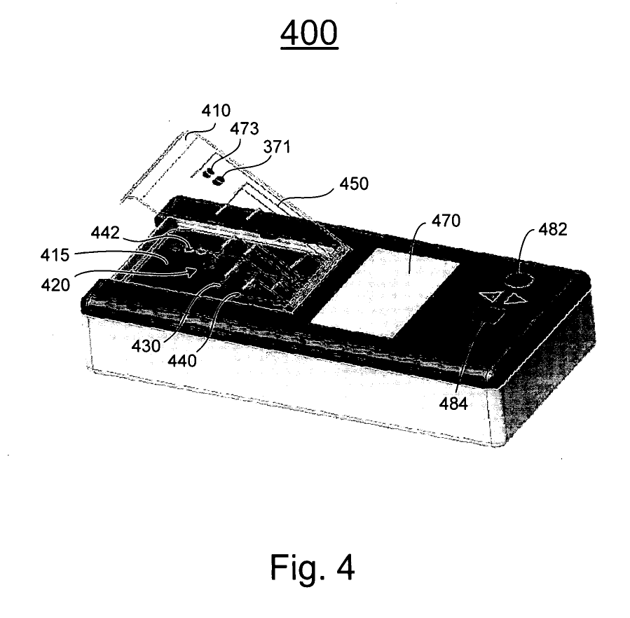

Referring to FIG. 4, a handheld flow cytometer LOC device 400 according to one embodiment of the present invention is shown schematically. The optical fibers 471 and 473 introducing the excitation lasers and the electrodes 450 are built into the cover lid 410 (only dot 471 and dot 473 are shown to indicate the fiber heads' positions. The remaining fibers are not shown for clarity). The tip of the detection optical fibers 471 and 473 are fixed at the surface of the chip-holding stage 418. To operate the device 400, first, the microfluidic flow cytometer chip 415 is placed on the chip-holding stage 418 which ensures the precise alignment between the optical fibers and the detection microchannel 442, and between the electrodes 440 and wells 430 of the microchannel structure 420. Then the sample and the buffer solution are loaded to the specific wells 430 by using a pipette. After that, the operator just needs to close the cover lid 410 and to press the button 482 to start the operation program. The chip 415 can be disposed after the test. The operation program is stored in a microprocessor chip (not shown) in the handheld LOC device 400. The status of the operation is shown on the LCD screen 470 of the handheld flow cytometer LOC device 400. The operation can be stopped by pushing the button 484 if necessary. Essential test results are shown on the screen 470 and the completed test results can be either displayed on the screen 470 or printed out. The complete testing data is temporarily saved in the device 400 and can be download to a computer or memory card for further analysis.

Another aspect of the present invention provides a method for counting and differentiating particles in a liquid medium of interest, where the liquid medium of interest contains one or more types of particles. In one embodiment, the method includes the steps of providing a microchannel structure on a first substrate; generating electrokinetically microfluidic flows to transport the liquid medium in the microchannel structure so as to differentiate the one or more types of particles of the liquid medium therein; and detecting the differentiated one or more types of particles of the liquid medium. In one embodiment, the microchannel structure is disclosed as above.

The step of generating electrokinetically microfluidic flows comprises the steps of placing an electrode into a corresponding port for each port of the microchannel structure; and individually applying voltages to each of the placed electrodes to generate desired electrokinetically microfluidic flows in the microchannel structure.

The detecting step comprises the steps of delivering at least one beam of laser to a microchannel at a position to illumine a particle passing through the position; collecting signals for a period of time, each signal associated with a particle passing through the position; and analyzing the collected signals to determine the number and type of the particles passing through the microchannel. In one embodiment, the signal associated with the particle comprises a fluorescent signal emitted from the particle in response to the illumination of the at least beam of laser.

According to the present invention, the flow cytometer lab-on-a-chip device is capable of detecting and/or treating a large number of different cells as required in clinical applications, and minimizes the total number of cells and particles to be counted. Minimizing the total number of to-be-counted events reduces the analysis time and the complexity of the optical detection system while increasing the accuracy. The flow cytometer lab-on-a-chip device in operation removes large cells such as granulocytes and monocytes, and small components such as platelets and the lysed red cells' debris, prior to counting CD4 and CD3 cells.

Another feature of such a flow cytometer lab-on-a-chip device is to provide the total number of CD4+ T lymphocytes, in addition to their percentages, in the sample of interest. Because monocytes can overlap lymphocytes in size and can also express low levels of CD4, they must be identified to avoid falsely elevated counts of CD4+ lymphocytes.

Other features of the flow cytometer lab-on-a-chip device include no external pump, no tubing and valves, no bulky optical detection instruments, and a low-cost disposable chip. Electrokinetic-microfluidic means to transport liquid and cells in microchannels requiring only the application of electrical fields via electrodes inserted in different wells. A portable multiple wavelength detection system is utilized by small diode lasers, Si-PIN detectors and optical fibers. Additionally, the flow cytometer lab-on-a-chip is made of PDMS and glass plates by a soft photolithography technique, no embedded waveguides or optical fibers is embedded into the chip, thereby, making the chip inexpensive and disposable.

These and other aspects of the present invention are further described below.

### Examples and Implementations of the Invention

Without intent to limit the scope of the invention, exemplary procudures and their related results according to the embodiments of the present invention are given below. Note again that titles or subtitles may be used in the examples for convenience of a reader, which in no way should limit the scope of the invention. Moreover, certain theories are proposed and disclosed herein; however, in no way they, whether they are right or wrong, should limit the scope of the invention.

### Separations of Cells in a Sample

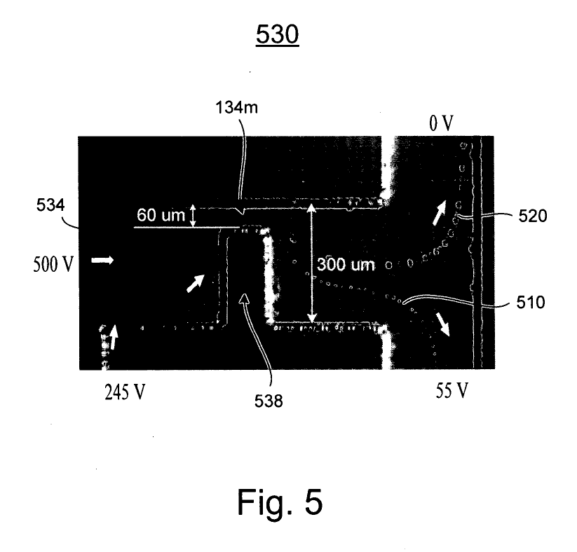

According the present invention, the cells in a sample (liquid) are separated by induced DC-DEP forces in a particle separation unit. FIG. 5 shows an image of trajectories 510 and 520 of polystyrene particles having sizes of about 6 μm and about 15 μm, separated by particle separation unit 500. The trajectories 510 and 520 of polystyrene particles are obtained by superimposing a series of sequential microscopy images. The microchannel 534 in this embodiment is about 300 μm in width and about 40 μm in depth (perpendicular to the paper). The narrow section 534m of the microchannel 534 is about 60 μm in width. The voltages applied to the first and second inlet ports and a first and second outlet ports are about 245 V, 500 V, 55 V and 0 V, respectively. When the mixed particles approach the narrow gap 534m from the side of the hurdle 538, the DC-DEP effect produces the best separation of particles. As explained above, when larger particles and smaller particles move closely over the corner of the hurdle 538 where the non-uniform electrical field gradient is the strongest, the larger particles are subject to a stronger DEP force and are pushed further away from the corner compared with smaller particles. Consequently the larger particles and smaller particles follow separate trajectories 520 and 510 after passing the hurdle 538.

### Flow Focusing

In one embodiment of the present invention, a flow cytometer is capable of focusing a cell-carrying stream so that only single cells are allowed to pass the sensing (detecting) point, and optically detecting a specific type of cell by detecting the fluorescent signal carried by each cell.

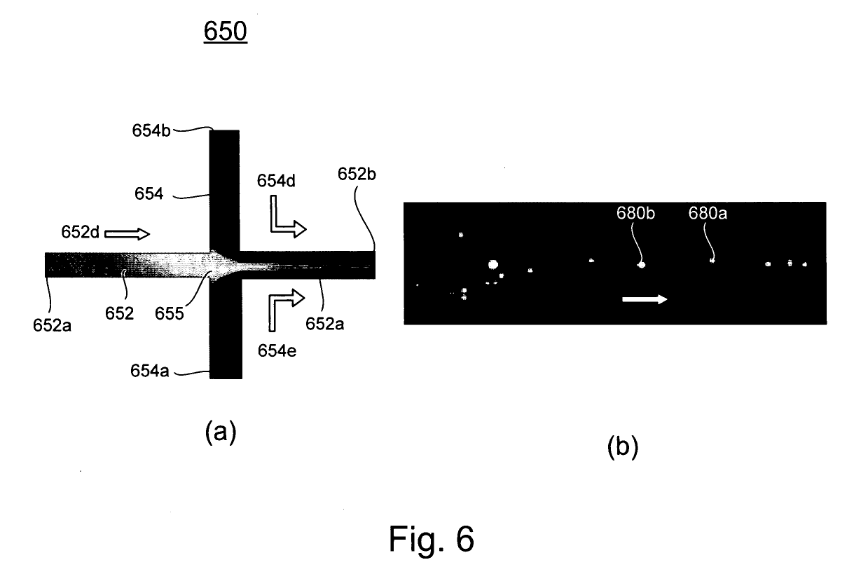

As shown in FIG. 6, a stream (flow) focusing system 650 according to one embodiment of the present invention is shown. The stream focusing system 650 has a cross-shaped microchannel structure having a horizontal microchannel 652 and a vertical microchannel 654 in fluid communication with the horizontal microchannel 652 through a junction 655 formed therein. The microchannel structure is filled with a buffer solution. One end 652a of the horizontal channel 652 of the microchannel structure is in fluid communication with a sample well filled with a buffer solution containing the cells to be detected, the other end 652b of the horizontal channel 652 is in fluid communication with a waste collection well. The ends 654a and 654b of the vertical microchannel 654 are respectively in fluid communication with two wells filled with a buffer solution. Four electrodes are inserted in these wells. When different voltages are applied to the four wells via the electrodes, the electrical fields generate electroosmotic flows in the microchannel structure. The electrical fields are applied in such a way that the three liquid streams 652d, 654d and 654e from the sample well and the two buffer wells flow towards the waste well, and they meet at the cross intersection (junction) 655. The electroosmotic flows in the microchannel structure are laminar flows and do not mix streams 652d, 654d and 654e. By adjusting the flow rates, i.e., adjusting the electrical potentials, the two side flows (buffer solution) 654d and 654e squeeze the central cell-carrying flow 652d to a desired size, thereby focusing the stream 652d. In this case, particles (cells) 680a and 680b singly pass through the detecting point.

According to the present invention, a set of four electrical potential values applied to the four wells is dependent from a specified main flow (the cell-carrying solution) rate and a specified cell size (the focused stream size). Controlling the flow field in the intersection region of the cross microchannel also depends on the liquid properties (e.g., viscosity and ionic concentration), the shape and the size of the intersection and the applied electrical fields. In one embodiment, this is achieved by developing a theoretical model that simulates accurately the flows and the focusing process. Such an experimentally verified model is then used to control the lab-on-a-chip flow cytometer operation via a computer program. A fluorescent image analysis system is used to visualize the flow focusing process near the intersection. The profile of the focused flow stream is measured. The prediction (the numerical simulation results) of such a flow focusing is verified by the experimental results [5-9].

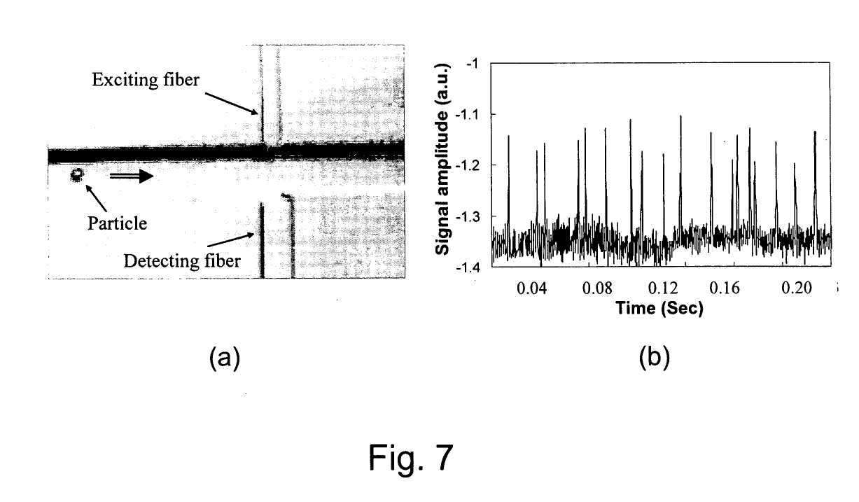

FIG. 6 shows the flow focusing images demonstrating the online counting of particles in a flow cytometer chip by using embedded optical fibers in the PDMS flow cytometer chip. A small size semiconductor laser and a Si-PIN detector are used for optical detection. The detection system allows an easy switch between two-fiber detection mode and one-fiber detection mode, and is capable of counting particles, measuring particle velocity and identifying particle sizes. FIG. 7 shows a pair of the embedded optical fibers on the opposite sides of a microchannel, and the particle counting data. By simply adding additional lasers of a different wavelength and additional Si-PIN photo detectors, this device can detect different wavelengths carried by different particles or cells.

### Operation of the Flow Cytometer LOC Device

Initial sample preparation processes: in one embodiment, the process is performed as follows: about 50 μl volumes of blood are mixed with about 50 μl of a red blood cell lysis buffer (Caltag, Burlingame, Calif.) to lyse the red blood cells, and then diluted with about 500 μl of de-ionized water, which this protocol fixes WBC in the sample, and lyses RBC. About 10 μl of this sample solution is loaded to the sample well (S in FIG. 1) on the chip by a micro-pipette. About 10 μl of the sample solution contains approximately 8,000 cells (granulocytes, monocytes, and lymphocytes) and approximately 100,000 small components (platelets, RBC debris, etc).

On-chip processes: (1) Removing cells larger than 10 μm by a DC-DEP technique. This is conducted by applying predetermined voltages to wells B1, S, C1 and C2, as shown in FIG. 1. This process reduces the total number of cells to be counted and thus reduces the time, the number of detection microchannels and the complexity of the optical detection system. It is noted that the sample solution contains approximately a total of 8,000 cells, and T lymphocytes are smaller than 10 μm. By removing the cells larger than 10 μm, the total number of to-be-counted cells is reduced by ⅔, to about 3,000 cells. (2) Removing components smaller than 4 μm (platelets, RBC debris, etc) by the DC-DEP technique. This is conducted by applying predetermined voltages to wells B2, C2, C3 and C4, as shown in FIG. 1. This separation is for two purposes. First, it is to reduce the total number of particles to be counted and hence reduce the time, reduce the number of detection microchannels and the complexity of the optical detection system. T lymphoctes are larger than 4 μm, and virtually all the debris components in a typical sample are less than 4 μm. By removing the small components (smaller than 4 μm), the total number of to-be-counted particles is dramatically reduced to 3,000 total cells, which is predominantly lymphocytes. Secondly, because some of these small components could carry the dyes by non-specific adsorption, removing these small components improves the reliability of the CD4 and CD3 counting. The separated cells (with a size range from 4 μm to 10 μm) are electrokinetically transported from well C4 to the flow focusing channel. (3) Focusing the flow and counting CD4− and CD3-positive cells by detecting the two different wavelengths of the specific dye-tagged antibodies in the detection microchannels. In one embodiment, a vertical optical detection method is used, i.e., using two optical fibers from the top of the PDMS to introduce the exciting laser beams, and two optical fibers underneath the glass plate to receive the emission light signals.

For the flow cytometer chip operation at step (1), the DC-DEP separation of larger cells takes approximately one minute to complete. The typical speed of particle electrokinetic motion in the microchannels is about 1000 μm/s. Of the approximate 108,000 particles (cells and small components) in the 10 μl sample, 100,000 of them are smaller than 4 μm. For example, if the narrowest section of the microchannel in the DC-DEP part is approximately 50 μm, considering that multiple particles are moving in parallel through the microchannel, approximately 2,000˜3,000 particles/second or 120,000˜180,000 particles/min are processed. The approximately 3,000 larger cells (>10 μM) out of the 108,000 particles (cells and the small components) can therefore be separated within one minute.

For the flow cytometer chip operation at step (2), the DC-DEP separation of small components takes approximately one minute to complete. The typical speed of particle electrokinetic motion in the microchannels is about 1000 μm/s. About 97% of the particles are the small components with size smaller than 4 μm. Since multiple particles are moving in parallel through the channel, approximately 2,000˜3,000 particles/second or 120,000˜180,000 particles/min are processed. Therefore approximately 100,000 small particles can therefore be separated within one minute.

After the separation, there are approximately 5,000 cells with a size ranging from 4 μm to 10 μm. It takes one minute to complete the counting of CD3 and CD4 cells from the 5,000 cells. Considering the cell electrokinetic speed is about 1000 μm/s, and the cells' size is less than 10 μm, more than 100 cells/s or more than 6000 cells/min pass the sensing point in a single microchannel.

### Detection of CD4+ T Lymphocytes

To facilitate staining and analysis of samples of interest, several protocols are available for whole blood staining of lymphocytes for surface marker expression. Whole blood is incubated with fluorochrome-conjugated monoclonal antibodies for about 30 minutes in the dark, and this is usually followed by RBC lysis and several washes. In one embodiment, a modified protocol that omits any washing steps is disclosed, which eliminates the need for an expensive centrifuge. In one exemplary study, it is shown that there is no discernible difference in the level of staining, or the amount of “debris” present in the sample, without washing. This lysis step preserves the WBC in the sample, and in three subjects there is no difference in the % values of CD4 or CD8 lymphocytes comparing whole blood staining to staining of peripheral blood mononuclear cells (PBMC) obtained after ficoll density centrifugation (FIG. 4).

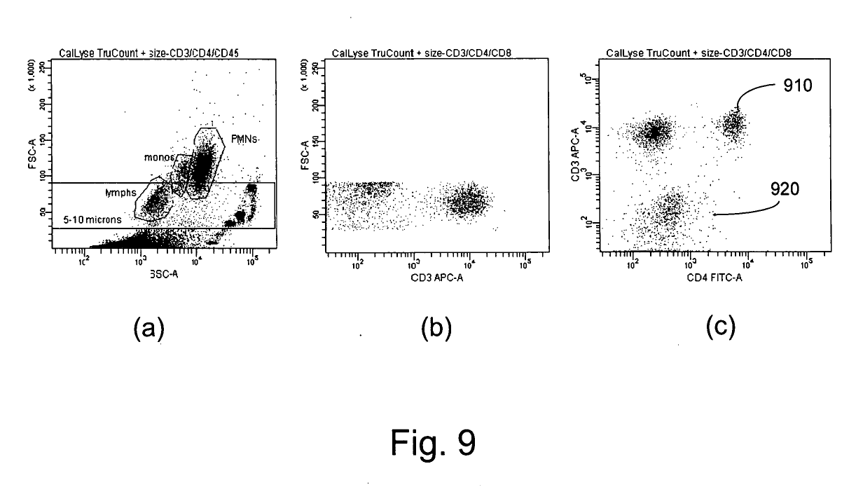

Any device that requires “counting” of individual cells needs to discriminate between an actual cell, and debris. Ideally, debris should be filtered prior to reaching the laser to minimize potential noise, e.g. autofluorescence from dead cell debris. As shown in FIG. 9, traditional gating strategies are used to illustrate this point. The total number of “events” recorded by the cytometer in the figure below is 85,026. Of those events, 12,431 events are larger than the lower line (approximately 4 microns, but this is not a precise value). The upper threshold (approximately 10 microns) is used to exclude the larger monocytes and polymorphonuclear leukocytes (PMNs). The size exclusion criteria remove 90% of PMNs and 70% of monocytes from the final analysis. This leaves approximately 4,873 events in the proper size range to include all the lymphocytes. In the proposed device, only particles of this size range will reach the lasers.

These cells is then evaluated for CD3, as shown in FIG. 9b, and CD4 expression. The final plot shown in FIG. 9c shows all events in the “4-10 micron” size range, and their expression of the CD3+ and CD4+ cell markers. These two colors easily discriminate the T cells from the monocytes (minimal CD3 staining, and CD4 dim) and PMNs (CD3 and CD4 negative).

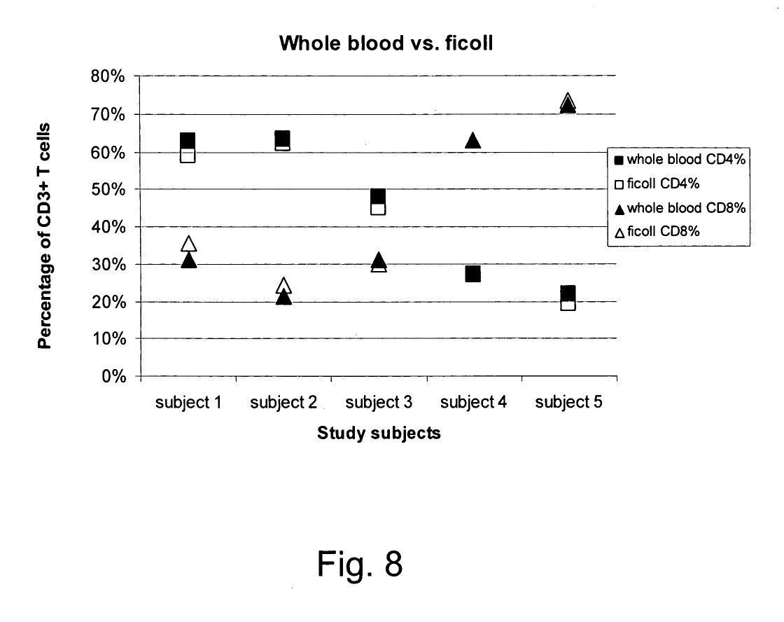

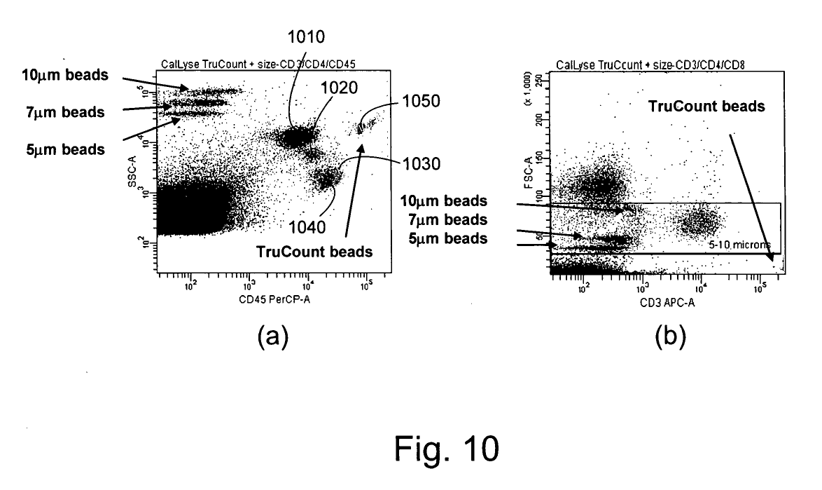

To determine the precision and accuracy of the device internally, samples need to be run in parallel. Since the miniature flow cytometer according to the present invention provide s a “single platform” method of counting T cells, a standard need to be designed for comparison. Referring to FIG. 8, it demonstrates the high concordance in the % of CD4 T cells derived from whole blood staining and PBMC isolated over ficoll, where the absolute CD4+ T cell number in whole blood samples is evaluated. The current standard for analysis is a “dual platform method”. Whole blood is run on a Coulter counter for evaluation of total lymphocytes (part of a CBC panel). The percentage of CD4+ T cells is evaluated by flow, and these two numbers are multiplied to give the total CD4+ T cell number. The present invention, among other things, provides a new standard that uses a “single platform” method. This is achieved by running a known number of standard beads in the sample. With this method, about 50 microliters of blood are added to a standardized tube with a known number of beads (49, 944 in this case). The absolute CD4 count is determined by the following formula: (number of CD3+ CD4+ events/number of beads counted)×(number of beads per tube/sample volume). Referring to FIG. 10, a relatively healthy HIV(+) individual is shown. The CD4+ T cell count was 449/mm³ (normal 400-1600). The actual value from the reference laboratory was 446/mm³.

The present invention, among other things, discloses an electrokinetic microfluidic flow cytometer lab-on-a-chip device that realizes multiple functions and/or processes for flow cytometry. The device utilizes a miniature laser-optical fiber based multiple wavelength detection system to count and differentiate particles in a liquid medium of interest.

The electrokinetic microfluidic LOC device of the present invention can find many applications in a wide spectrum of fields including, but not limited to, counting CD4 cells, proteomics and DNA analysis, drug development, chemical development, and so on.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

[1]. Tung Y C, Zhang M, Lin C T, Kurabayashi K, Skerlos S J. PDMS-based opto-fluidic micro flow cytometer with two-color, multi-angle fluorescence detection capability using PIN photodiodes. Sensors and Actuators B: Chemical. 2004;98:356-67.
[2]. Fu L M, Yang R J, Lin C H, Pan Y J, Lee G B. Electrokinetically driven micro flow cytometers with integrated fiber optics for on-line cell/particle detection. Analytica Chimica Acta. 2004;507:163-69.
[3]. Rodriguez W R, Christodoulides N, Floriano P N, Graham S, Mohanty S, Dixon M, Hsiang M, Peter T, Zavahir S, Thior I, Romanovicz D, Bernard B, Goodey A P, Walker B D, McDevitt J T. A microchip CD4 counting method for HIV monitoring in resource-poor settings. PLoS Med. 2005;2:e182.
[4]. Xiang Q, Xuan X, Xu B., Li D. Multi-functional Particle Detection with Embedded Optical Fibers in a Poly(dimethylsiloxane) Chip. Instrumentation Sci & Tech. 2005;33:597-607.
[5]. Ren L, Li D. Theoretical studies of microfluidic dispensing processes. J Colloid Interface Sci. 2002;254:384-95.
[6]. Ren L, Sinton D, Li D. Numerical simulation of microfluidic injection processes in crossing microchannels. Journal of Micromechanics and Microengineering. 2003;739-47.
[7]. Sinton D, Ren L, Xuan X, Li D. Effects of liquid conductivity differences on multi-component sample injection, pumping and stacking in microfluidic chips. Lab Chip. 2003;3:173-79.
[8]. Sinton D, Ren L, Li D. A dynamic loading method for controlling on-chip microfluidic sample injection. J Colloid Interface Sci. 2003;266:448-56.
[9]. Sinton D, Ren L, Li D. Visualization and numerical modelling of microfluidic on-chip injection processes. J Colloid Interface Sci. 2003;260:431-39.
[10]. Pohl H A. Dielectrophoresis. London: Cambridge University Press; 1978.
[11]. Lapizco-Encinas B H, Simmons B A, Cummings E B, Fintschenko Y. Insulator-based dielectrophoresis for the selective concentration and separation of live bacteria in water. Electrophoresis. 2004;25:1695-704.
[12]. Kang K H, Kang Y, Xuan X, Li D. Continuous separation of microparticles by size with Direct current-dielectrophoresis. Electrophoresis. 2006;27:694-702.
[13]. Duffy D C, Schueller O J A, Brittain S T, Whitesides G M. Rapid prototyping of microfluidic switches in poly(dimethyl siloxane) and their actuation by electro-osmotic flow. Journal of Micromechanics and Microengineering. 1999;211-17.
[14]. Xuan X, Xu B, Li D. Accelerated particle electrophoretic motion and separation in converging-diverging microchannels. Anal Chem. 2005;77:4323-28.
[15]. Kang K H, Li D. Dielectric Force and Relative Motion between Two Spherical Particles in Electrophoresis. Langmuir. 2006;22:1602-8.
[16]. McMullin J N, Qiao H, Goel S, Ren C L, Li D. Integrated optical measurement of microfluid velocity. Journal of Micromechanics and Microengineering. 2005;1810-1816.
[17]. Xiang Q, Hu G, Gao Y, Li D. Miniaturized immunoassay microfluidic system with electrokinetic control. Biosens Bioelectron. 2005.
[18]. Xuan X, Raghibizadeh S, Li D. Wall effects on electrophoretic motion of spherical polystyrene particles in a rectangular poly(dimethylsiloxane) microchannel. J Colloid Interface Sci. 2005.
[19]. Xuan X, Ye C, Li D. Near-wall electrophoretic motion of spherical particles in cylindrical capillaries. J Colloid Interface Sci. 2005;289:286-90.
[20]. Xuan X, Li D. Focused electrophoretic motion and selected electrokinetic dispensing of particles and cells in cross-microchannels. Electrophoresis. 2005;26:3552-60.
[21]. Ye C, Li D. Electrophoretic Motion of a Sphere in a Microchannel under the Gravitational Field. J Colloid Interface Sci. 2002;251:331-38.
[22]. Ye C, Sinton D, Erickson D, Li D. Electrophoretic Motion of a Circular Cylindrical Particle in a Circular Cylindrical Microchannel. Langmuir. 2002;18:9095-101.
[23]. Ye C, Li D. 3-D transient electrophoretic motion of a spherical particle in a T-shaped rectangular microchannel. J Colloid Interface Sci. 2004;272:480-488.
[24]. Ye C, Li D. Electrophoretic motion of two spherical particles in a rectangular microchannel. Microfluidics and Nanofluidics. 2004;1:52-61.
[25]. Ye C, Xuan X, Li D. Eccentric electrophoretic motion of a sphere in circular cylindrical microchannels. Microfluidics and Nanofluidics. 2005; 1:234-41.
[26]. Young E W, Li D. Dielectrophoretic force on a sphere near a planar boundary. Langmuir. 2005;21:12037-46.
[27]. Steger, J. L., Dougherty, F. C., and Benek, J. A. A Chimera Grid Scheme. 1983. ASME Mini-Symposium on Advances in Grid Generation. Ref Type: Conference Proceeding
[28]. Erickson D, Liu X, Krull U, Li D. Electrokinetically controlled DNA hybridization microfluidic chip enabling rapid target analysis. Anal Chem. 2004;76:7269-77.
[29]. Erickson D, Liu X, Venditti R, Li D, Krull U J. Electrokinetically based approach for single-nucleotide polymorphism discrimination using a microfluidic device. Anal Chem. 2005;77:4000-4007.
[30]. Gao Y, Hu G, Lin F Y, Sherman P M, Li D. An electrokinetically-controlled immunoassay for simultaneous detection of multiple microbial antigens. Biomed Microdevices. 2005;7:301-12.
[31]. Gao Y, Lin F Y H, Hu G, Sherman P M, Li D. Development of a novel electrokinetically driven microfluidic immunoassay for the detection of Helicobacter pylori. Analytica Chimica Acta. 2005;543:109-16.
[32]. Hu G, Gao Y, Sherman P, Li D. A microfluidic chip for heterogeneous immunoassay using electrokinetical control. Microfluidics and Nanofluidics. 2005;1:346-55.

What is claimed is:

1. A flow cytometer for counting and differentiating particles in a liquid medium of interest, comprising:
a. a first substrate having a first surface and an opposite, second surface defining a body portion therebetween;
b. a microchannel structure formed in the body portion of the first substrate, the microchannel structure comprising a first particle separation unit, a second particle separation unit, and a flow focusing unit,
wherein each of the first and second particle separation units has a first, and second inlet ports, and a first, second and third outlet ports and, and a first, second and third microchannels, each of the first, second and third microchannels formed with a first open end, an opposite, second open end, respectively.
wherein the first microchannel is in fluid communication with the first inlet port and the second microchannel through the first and second open ends and, respectively, thereby forming a first junction of the first and second microchannels;
wherein the second microchannel is in fluid communication with the second inlet port and the third microchannel through the first and second open ends and, respectively, thereby forming a second junction of the second and third microchannels; and
wherein the third microchannel is in fluid communication with the first and second outlet ports and through the first and second open ends, respectively;
wherein the flow focusing unit has a first, second and third inlet ports, an outlet port, and a first and second microchannel, each of the first and second microchannels formed with a first open end, an opposite, second open end, respectively,
wherein the first microchannel is in fluid communication with the first inlet port and the outlet port through its first and second open ends, respectively;
wherein the second microchannel is in fluid communication with the second and third inlet ports and through its first and second open ends, respectively; and
wherein the first and second microchannels are in fluid communication with each other through a junction formed therein;
wherein the first inlet port of the second particle separation unit coincides with one of the first and second outlet ports of the first particle separation unit, and the first inlet port of the flow focusing unit coincides with one of the first and second outlet ports of the second particle separation unit;
c. a fluid control member configured to control flow of the liquid medium in the microchannel structure; and
d. an optical detection unit coinfigured to count and differentiate particles in the liquid medium.

2. The flow cytometer of claim 1, wherein the first junction formed in each of the first and second particle separation units divides the second microchannel into a first branch and a second branch, wherein the first branch is between the first open end of the second microchannel and the first junction, and wherein the second branch is between the first junction and the second open end of the second microchannel;
wherein the second junction formed in each of the first and second particle separation units divides the third microchannel into a first branch and a second branch, wherein the first branch is between the first open end of the third microchannel and the first junction, and wherein the second branch is between the first junction and the second open end of the third microchannel; and
wherein the junction formed in the flow focusing unit divides each of the first and second microchannels into a first branch and a second branch, wherein the first branch of each of the first and second microchannels is between the first open end of the corresponding microchannel and the junction, and wherein the second branch of each of the first and second microchannels is between the junction and the second open end of the corresponding microchannel.

3. The flow cytometer of claim 1, wherein each microchannel of the first and second particle separation units and the flow focusing unit is formed with a first side wall and an opposite, second side wall defining a corresponding channel width therebetween.

4. The flow cytometer of claim 3, wherein each channel width is in a range of about 0.1-1,000 μm, preferable in a range of about 1-500 μm.

5. The flow cytometer of claim 3, wherein each of the first and second particle separation units further has a hurdle protruded inwards from the first side wall of the second branch of the second microchannel.

6. The flow cytometer of claim 5, wherein the hurdle has a cross-sectional geometric shape with a height, h, wherein the cross-sectional geometric shape is selected from the group consisted of a triangle, a square, a rectangle, a semi-circle and a polygon, and the height h is less than the width, w2, of the second microchannel so as to allow particles in the liquid medium of interest to pass through.

7. The flow cytometer of claim 5, wherein the hurdle is formed of a dielectric material.

8. The flow cytometer of claim 1, further comprising a second substrate having a first surface and an opposite, second surface, wherein the second substrate is bonded to the first substrate such that the first surface of the second substrate is substantially in contact with the second surface of the first substrate, thereby sealing the microchannel structure formed in the body portion of the first substrate.

9. The flow cytometer of claim 8, wherein the fluid control member comprises:
a. a plurality of electrodes, each electrode placed in a corresponding port of the first and second particle separation units and the flow focusing unit; and
b. a power source electrically coupled with the plurality of electrodes for individually applying voltages to each of the plurality of electrodes so as to generate desired electrokinetically microfluidic flows in the first and second particle separation units, respectively, and the flow focusing unit for separating and transporting the particles in the liquid medium of interest.

10. The flow cytometer of claim 9, wherein the fluid control member further comprises a controller in communication with the power source and the plurality of electrodes for regulating voltages applied to each of the plurality of electrodes.

11. The flow cytometer of claim 10, wherein in operation, the voltages are applied to the electrodes placed in the first and second inlet ports and the first and second outlet ports of each of the first and second particle separation units, respectively, such that the generated electrokinetically microfluidic flows cause
a liquid medium of interest introduced to the first inlet port and a buffer solution introduced to the second inlet port to move along the first microchannel and the first branch of the second microchannel, respectively, towards the first junction, and to merge into a stream of fluid therein;
the merged stream of fluid to move along the second branch of the second microchannel towards and through the hurdle and towards the second junction, and to separate into a first and second streams of fluid therein; and
the separated first and second streams of fluid to move along the first and second branches of the third microchannels towards the first and second outlet ports, respectively, of the corresponding particle separation unit,
wherein the separated first stream of fluid contains particles that are substantially different from these contained in the separated second stream of fluid.

12. The flow cytometer of claim 11, wherein in operation, the voltages are applied to the electrodes placed in the first to third inlet ports and the outlet port of the flow focusing unit, respectively, such that the generated electrokinetically microfluidic flows cause
a particle-carrying flow from the first inlet port, a first buffer solution flow from the second inlet port, a second buffer solution flow from the third inlet port to move towards and meet at the junction, and to move towards the outlet port; and
the first buffer solution flow and the second buffer solution flow to squeeze the particle-carrying flow to a desired size in the second branch of the first microchannel, thereby focusing the particle-carrying flow such that each particle moves singly along the second branch of the first microchannel towards the outlet port.

13. The flow cytometer of claim 12, wherein the optical detection unit comprises:
a. one or more input optical fibers, each input optical fiber positioned over the second branch of the first microchannel of the flow focusing unit from the first substrate for delivering a corresponding beam of laser thereto to illumine the particles in the focused stream of fluid passing therethrough;
b. one or more output optical fibers, each output optical fiber positioned opposite to a corresponding input optical fiber from the second substrate such that when a particle passes through a position to which a beam of laser is delivered from the corresponding input optical fiber, the output optical fiber receives a signal associated with the particle; and
c. a plurality of detectors coupled with the one or more output optical fibers for recording signals received from the one or more output optical fibers, wherein the recorded signals are usable for counting and differentiating the particles passing through the second branch of the first microchannel of the flow focusing unit.

14. The flow cytometer of claim 13, wherein each of the one or more input optical fibers and the one or more output optical fibers comprises a multimode optical fiber that has a diameter in a range of about 10-200 μm.

15. The flow cytometer of claim 13, wherein the signal associated with the particle comprises a fluorescent signal emitted from the particle in response to the illumination of the beam of laser.

16. The flow cytometer of claim 15, wherein the optical detection unit further comprises a plurality of filters, each filter coupled between the one or more output optical fibers and one of the plurality of detectors, respectively.

17. The flow cytometer of claim 8, wherein each of the first and second substrates is formed of a corresponding dielectric material.

18. The flow cytometer of claim 17, wherein the first substrate is formed of polydimethylsiloxane (PDMS), and the second substrate is formed of glass, respectively.

19. The flow cytometer of claim 1, wherein the liquid medium of interest comprises a biological fluid of a living subject, wherein the biological fluid comprises blood or urine, and wherein the blood or urine comprises one or more types of particles or cells.

20. The flow cytometer of claim 19, wherein the one or more types of cells are differentiatable by their sizes, functions or a combination of them.

21. The flow cytometer of claim 19, wherein the one or more types of cells comprise CD4+ cells, and/or CD3+ cells.

22. The flow cytometer of claim 21, wherein the CD4+ cells and CD3+ cells are labeled with a first and second antibodies, respectively, wherein the first and second antibodies are excited with light of different wavelengths.

23. The flow cytometer of claim 19, wherein the one or more types of cells are associated with a disease.

24. A flow cytometer, comprising:
a. a microchannel structure adapted for transporting a fluid medium containing one or more types of particles;
b. means for generating electrokinetically microfluidic flows to transport the fluid medium in the microchannel structure so as to differentiate the one or more types of particles in the fluid medium; and
c. an optical detection system configured to detect the differentiated one or more types of particles of the fluid medium.

25. The flow cytometer of claim 24, wherein the microchannel structure comprises at least one particle separation unit, wherein the at least one particle separation unit comprises at least one inlet port, a first and second outlet forts, and at least one channel in fluid communication with the at least one inlet port and the first and second outlet ports, wherein the at least one microchannel is formed with at least one side wall and a hurdle protruded inwards from the at least one sidewall such that when the fluid medium is introduced into the at least one microchannel and passes through the hurdle, the one or more types of particles are dielectrophoretically differentiated into a first and second groups of particles in accordance with their sizes, wherein the first and second groups of particles move towards the first and second outlet ports, respectively.

26. The flow cytometer of claim 25, wherein the hurdle has a cross-sectional geometric shape selected from the group consisted of a triangle, a square, a rectangle, a semi-circle and a polygon.

27. The flow cytometer of claim 25, wherein the microchannel structure further comprises a flow focusing unit in fluid communication with the at least one particle separation unit, wherein the flow focusing unit comprises at least one inlet port, an outlet port and at least one microchannel in fluid communication with the at least one inlet port and the outlet port, and wherein when one of the first and second groups of particles received in a corresponding outlet port of the at least one particle separation unit is introduced to the at least one microchannel from the at least one input port, each particle moves singly along the at least one microchannel towards the outlet port.

28. The flow cytometer of claim 27, wherein the optical detection system comprises:
a. one or more input optical fibers, each input optical fiber positioned over the at least one microchannel of the flow focusing unit 350 for delivering a corresponding beam of laser thereto to illumine the particles passing therethrough;
b. one or more output optical fibers, each output optical fiber positioned opposite to a corresponding input optical fiber such that when a particle passes through a position to which a beam of laser is delivered from the corresponding input optical fiber, the output optical fiber receives a signal associated with the particle; and
c. a plurality of detectors coupled with the one or more output optical fibers for recording signals received from the one or more output optical fibers, wherein the recorded signals are usable for counting and differentiating the particles passing through the second branch of the first microchannel of the flow focusing unit.

29. The flow cytometer of claim 24, wherein the fluid medium comprises a biological fluid of a living subject, wherein the biological fluid comprises blood or urine, and wherein the blood or urine comprises one or more types of particles or cells.

30. The flow cytometer of claim 29, wherein the one or more types of cells are differentiatable by their sizes, functions or a combination of them.

31. The flow cytometer of claim 29, wherein the one or more types of cells comprise CD4+ cells, and/or CD3+ cells.

32. The flow cytometer of claim 31, wherein the CD4+ cells and CD3+ cells are labeled with a first and second antibodies, respectively, wherein the first and second antibodies are excited with light of different wavelengths.

33. The flow cytometer of claim 29, wherein the one or more types of cells are associated with a disease.

34. A method for counting and differentiating particles in a liquid medium of interest, wherein the liquid medium of interest contains one or types of particles, comprising the steps of:
a. providing a microchannel structure on a first substrate;
b. generating electrokinetically microfluidic flows to transport the liquid medium in the microchannel structure so as to differentiate the one or more types of particles in the liquid medium therein; and
c. detecting the differentiated one or more types of particles in the liquid medium.

35. The method of claim 34, wherein the microchannel structure comprises at least one particle separation unit, wherein the at least one particle separation unit comprises a first and second inlet ports, a first and second outlet ports, and a first to third microchannels, each of the first to third microchannels formed with a first open end, an opposite, second open end, and a first side wall and an opposite, second side wall defining a corresponding width therebetween,
wherein the first microchannel is in fluid communication with the first inlet port and the second microchannel through the first and second open ends, respectively, thereby forming a first junction that divides the second microchannel into a first branch and a second branch, wherein the first branch is between the first open end and the first junction, and the second branch is between the first junction and the second open end;
wherein the second microchannel is in fluid communication with the second inlet port and the third microchannel through its first and second open ends, respectively, thereby forming a second junction that divides the third microchannel into a first branch and a second branch, wherein the first branch is between the first open end and the second junction, and the second branch is between the second junction and the second open end; and
wherein the third microchannel is in fluid communication with the first and second outlet ports through its first and second open ends, respectively.

36. The method of claim 35, wherein the at least one particle separation units further has a hurdle protruded inwards from the first side wall of the second branch of the second microchannel.

37. The method of claim 36, wherein the hurdle has a cross-sectional geometric shape with a height, h, wherein the cross-sectional geometric shape is selected from the group consisted of a triangle, a square, a rectangle, a semi-circle and a polygon, and the height h is less than the width, w2, of the second microchannel so as to allow one or more types of particles of the liquid medium to pass through the second branch of the second microchannel.

38. The method of claim 36, wherein the microchannel structure further comprises a flow focusing unit in fluid communication with the at least one particle separation unit, wherein the flow focusing unit further has a first, second and third inlet ports, an outlet port, and a first and second microchannels, each of the first and second microchannels formed with a first open end, an opposite, second open end, and a first side wall and an opposite, second side walls defining a width therebetween,
wherein the first microchannel is in fluid communication with the first inlet port and the outlet port through its first and second open ends, respectively;
wherein the second microchannel is in fluid communication with the second and third inlet ports through its first and second open ends, respectively; and
wherein the first and second microchannels are in fluid communication with each other through a junction formed therein, and the junction divides each of the first and second microchannels into a first branch and a second branch, wherein the first branch of each of the first and second microchannels is between the first open end of the corresponding microchannel and the junction, and wherein the second branch of each of the first and second microchannels is between the junction and the second open end of the corresponding microchannel

39. The method of claim 38, wherein the step of generating electrokinetically microfluidic flows comprises the steps of:
a. placing an electrode into a corresponding port for each of the first and second inlet ports and the first and second outlet ports of the at least one particle separation unit and the first, second and third inlet ports and the outlet port of the flow focusing unit; and
b. individually applying voltages to each of the placed electrodes to generate electrokinetically microfluidic flows in the at least one particle separation unit and the flow focusing unit.

40. The method of claim 39, wherein the generated electrokinetically microfluidic flows in the at least one particle separation unit cause
a liquid medium of interest introduced to the first inlet port and a buffer solution introduced to the second inlet port to move along the first microchannel and the first branch of the second microchannel, respectively, towards the first junction, and to merge into a stream of fluid therein;
the merged stream of fluid to move along the second branch of the second microchannel towards and through the hurdle and towards the second junction, and to separate into a first and second streams of fluid therein; and
the separated first and second streams of fluid to move along the first and second branches of the third microchannels towards the first and second outlet ports, respectively, of the corresponding particle separation unit,
wherein the separated first stream of fluid contains particles that are substantially different from these contained in the separated second stream of fluid.

41. The method of claim 40, wherein the generated electrokinetically microfluidic flows in the flow focusing unit cause
a particle-carrying flow from the first inlet port, a first buffer solution flow from the second inlet port, a second buffer solution flow from the third inlet port to move towards and meet at the junction, and to move towards the outlet port; and
the first buffer solution flow and the second buffer solution flow to squeeze the particle-carrying flow to a desired size in the second branch of the first microchannel, thereby focusing the particle-carrying flow such that each particle moves singly along the second branch of the first microchannel towards the outlet port.

42. The method of claim 41, wherein the detecting step comprises the steps of:
a. delivering at least one beam of laser to the second branch of the first microchannel of the flow focusing unit at a position to illumine a particle passing through the position;
b. collecting signals for a period of time, each signal associated with a particle passing through the position; and
c. analyzing the collected signals to determine the number and type of the particles passing through the second branch of the first microchannel of the flow focusing unit.

43. The method of claim 42, wherein the signal associated with the particle comprises a fluorescent signal emitted from the particle in response to the illumination of the at least beam of laser.

44. The method of claim 34, wherein the liquid medium of interest comprises a biological fluid of a living subject, wherein the biological fluid comprises blood or urine, and wherein the blood or urine comprises one or more types of particles or cells.

45. The method of claim 44, wherein the one or more types of cells are differentiatable by their sizes, functions or a combination of them.

46. The method of claim 44, wherein the one or more types of cells comprise CD4+ cells, and/or CD3+ cells.

47. The method of claim 46, wherein the CD4+ cells and CD3+ cells are labeled with a first and second antibodies, respectively, wherein the first and second antibodies are excited with light of different wavelengths.

48. The method of claim 47, wherein the one or more types of cells are associated with a disease.

49. A flowcytometer configured to perform the method of claim 34.
